(19) United States
(12) Patent Application Publication
(10) Pub. No.: US 2024/0238502 A1
(43) Pub. Date: Jul. 18, 2024

(54) LOW PROFILE DUAL FLUID INSTILLATION AND REMOVAL BRIDGE SYSTEM WITH FLUID DELIVERY AND PRESSURE SENSING CAPABILITIES

(71) Applicant: KCI Manufacturing Unlimited Company, Westmeath (IE)

(72) Inventors: James Killingworth SEDDON, Berkshire (GB); Benjamin Andrew PRATT, Berkshire (GB); Thomas Alan EDWARDS, Berkshire (GB); Justin RICE, San Antonio, TX (US)

(21) Appl. No.: 18/289,834

(22) PCT Filed: May 11, 2022
(86) PCT No.: PCT/IB2022/054373

§ 371 Date: Nov. 7, 2023

Related U.S. Application Data
(60) Provisional application No. 63/188,210, filed on May 13, 2021.

(51) Int. Cl. A61M 1/00

(52) U.S. Cl. CPC A61M 1/913; A61M 1/915; A61M 1/92; A61M 1/966; A61M 2207/00

(57) ABSTRACT

An apparatus for managing fluid from a tissue site may include a first portion and a second portion configured to be independently fluidly coupled to a tissue interface. The first portion can comprise a first end, a second end, a first fluid pathway extending from the first end to the second end, and a first plurality of features projecting into the first fluid pathway. The second portion can comprise a first end and a second end. The second portion can include a second fluid pathway, a third fluid pathway, and a fourth fluid pathway formed along a length of the second portion. The portion can also include a second plurality of features, a third plurality of features, and a fourth plurality of features projecting into the second fluid pathway, the third fluid pathway, and the fourth fluid pathway, respectively. Other apparatuses and methods are disclosed.

### CROSS-REFERENCE TO RELATED APPLICATIONS

This application claims the benefit of priority to U.S. Provisional Application No. 61/188,210, filed on May 13, 2021, which is incorporated herein by reference in its entirety.

### TECHNICAL FIELD

The invention set forth in the appended claims relates generally to tissue treatment systems and more particularly, but without limitation, to apparatuses and methods for providing negative-pressure therapy and instillation therapy.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and instillation therapy are widely known, improvements to therapy systems, components, and processes may benefit healthcare providers and patients.

### BRIEF SUMMARY

New and useful systems, apparatuses, and methods for treating tissue in a negative-pressure therapy environment are set forth in the appended claims. Illustrative embodiments are also provided to enable a person skilled in the art to make and use the claimed subject matter.

For example, in some embodiments, an apparatus for managing fluid from a tissue site may comprise a first portion and a second portion configured to be independently fluidly coupled to a tissue interface. The first portion can comprise a first end configured to be fluidly coupled to the tissue interface, a second end configured to be fluidly coupled to a first conduit, and a first fluid pathway extending from the first end to the second end. In some embodiments, the first portion can also include a first plurality of features projecting into the first fluid pathway. The second portion can comprise a first end configured to be fluidly coupled to the tissue interface and a second end configured to be fluidly coupled to a second conduit. The second portion can also include a second fluid pathway formed along a length of the second portion, a third fluid pathway formed along the length of the second portion, and a fourth fluid pathway formed along the length of the second portion. The second fluid pathway can comprise a second plurality of features projecting into the second fluid pathway. The third fluid pathway can comprise a third plurality of features projecting into the third fluid pathway. The fourth fluid pathway can comprise a fourth plurality of features projecting into the fourth fluid pathway. In some embodiments, the first portion and the second portion can each comprise a first layer and a second layer. In some embodiments, the first end of the first portion may further comprise an aperture covered by a removable cover layer.

Alternatively, other example embodiments of an apparatus for managing fluid from a tissue site may comprise a first bridge and a second bridge. The first bridge may have a first layer and a second layer. The first layer can include a polymeric film having an outer surface, an inner surface, and a first plurality of features extending from the inner surface. The second layer can include a polymeric film having an outer surface and an inner surface. The inner surface of second layer can be coupled to the first layer and cover the first plurality of features to form a first sealed space with the inner surface of the first layer. A first plurality of flow channels can be within the first sealed space. The second layer can also have an aperture configured to fluidly couple the first sealed space to the tissue site. In some embodiments, a first bridge conduit can be fluidly coupled to the first plurality of flow channels.

The second bridge may have a third layer and a fourth layer. The third layer can include a polymeric film and a second plurality of surface features extending from a surface of the third layer. The fourth layer can include a polymeric film and be coupled to the third layer to cover the second plurality of surface features and form a second sealed space between the third layer and the fourth layer. In some embodiments, the second bridge can also have a first barrier and a second barrier coupled between the third layer and the fourth barrier. The first barrier and the second barrier can define a second plurality of flow channels, a third plurality of flow channels, and a fourth plurality of flow channels. The second plurality of flow channels can be formed in the second sealed space between the first barrier and the second barrier. The third plurality of flow channels can be formed in the second sealed space between the first barrier and a first seal formed between a first portion of the third layer and a first portion of the fourth layer. The fourth plurality of flow channels can be formed in the second sealed space between the second barrier and a second seal formed between a second portion of the third layer and a second portion of the fourth layer. The third plurality of flow channels and the fourth plurality of flow channels can be outboard of the second plurality of flow channels. In some embodiments, a second bridge conduit can be fluidly coupled to the second plurality of flow channels and at least one sensing conduit may be fluidly coupled to the third plurality of flow channels and the fourth plurality of flow channels.

A method of manufacturing an apparatus for managing fluid from a tissue site is also described herein, wherein some example embodiments include forming a first bridge and forming a second bridge. Forming the first bridge can comprise providing a first layer including a polymeric film having an outer surface, an inner surface, and a first plurality of features extending from the inner surface; providing a second layer including a polymeric film having an outer surface and an inner surface; and coupling the inner surface of the second layer to the first layer. Coupling the inner surface of the second layer to the first layer can further include covering the first plurality of features to form a first sealed space with the inner surface of the first layer and a first plurality of flow channels within the first sealed space. In some embodiments, the second layer can have an aperture configured to fluidly couple the first sealed space to the tissue site. Forming the first bridge can also include fluidly coupling a first bridge conduit to the first plurality of flow channels.

Forming the second bridge may comprise providing a third layer including a polymeric film and a second plurality of features extending from a surface of the third layer, providing a fourth layer including a polymeric film, and coupling the fourth layer to the third layer to cover the second plurality of features and form a second sealed space between the third layer and the fourth layer. Forming the second bridge can also include forming a first barrier and a second barrier between the third layer and the fourth layer. The first barrier and the second barrier can define a second plurality of flow channels, a third plurality of flow channels, and a fourth plurality of flow channels. The second plurality of flow channels can be in the second sealed space between the first barrier and the second barrier. The third plurality of flow channels can be in the second sealed space between the first barrier and a first seal formed between a first portion of the third layer and a first portion of the fourth layer. The fourth plurality of flow channels can be in the second sealed space between the second barrier and a second seal formed between a second portion of the third layer and a second portion of the fourth layer. The third plurality of flow channels and the fourth plurality of flow channels can be outboard of the second plurality of flow channels. Additionally, forming a second bridge may further include fluidly coupling a second bridge conduit to the second plurality of flow channels and fluidly coupling at least one sensing conduit to the third plurality of flow channels and the fourth plurality of flow channels.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment and instillation treatment in accordance with this specification;

FIG. 2 is a schematic diagram of an example embodiment of the therapy system of FIG. 1, illustrating additional details that may be associated with some embodiments;

FIG. 3 is a schematic diagram of a dressing interface of FIG. 2, illustrating additional details that may be associated with some embodiments;

FIG. 4 is a segmented perspective view of a first bridge of the dressing interface of FIG. 3, illustrating additional details that may be associated with some embodiments;

FIG. 5 is a segmented perspective view of a second bridge of the dressing interface of FIG. 3, illustrating additional details that may be associated with some embodiments;

FIG. 6A is a cross-sectional view taken of a second applicator along line 6A-6A in FIG. 5, illustrating additional details that may be associated with some embodiments;

FIG. 6B shows a section view of the second applicator taken along line 6B-6B in FIG. 5, illustrating additional details that may be associated with some embodiments.

FIG. 6C is a section view of another example of the second applicator taken along line 6C-6C in FIG. 5, illustrating additional details that may be associated with some embodiments;

FIG. 7 is a plan view of a first layer of the first bridge, illustrating additional details that may be associated with some embodiments of the dressing interface of FIG. 3;

FIG. 7A is a section view of the first layer of the first bridge taken along line 7A-7A of FIG. 7, illustrating additional details that may be associated with some embodiments;

FIG. 7B is a section view of the first layer of the first bridge taken along line 7B-7B of FIG. 7, illustrating additional details that may be associated with some embodiments;

FIG. 8 is a plan view of another example of the first layer of the first bridge that may be associated with some embodiments of the dressing interface of FIG. 3;

FIG. 8A is a section view of the first layer of the first bridge taken along line 8A-8A of FIG. 8, illustrating additional details that may be associated with some embodiments;

FIG. 8B is a section view of the first layer of the first bridge taken along line 8B-8B of FIG. 8, illustrating additional details that may be associated with some embodiments;

FIG. 9 is a top view of a portion of the second bridge of the dressing interface of FIG. 3;

FIG. 9A is a section view of the second bridge taken along the line 9A-9A of FIG. 9, illustrating additional details that may be associated with some embodiments;

FIG. 9B is a section view of the second bridge taken along line 9B-9B of FIG. 9, illustrating additional details that may be associated with some embodiments;

FIG. 10 is an assembly view of the first bridge of the dressing interface of FIG. 3, illustrating additional details that may be associated with some embodiments;

FIG. 11 is an assembly view of the second bridge of the dressing interface of FIG. 3, illustrating additional details that may be associated with some embodiments;

FIG. 12 is a schematic diagram of another dressing interface, illustrating additional details that may be associated with some embodiments of the therapy system of FIG. 1;

FIG. 13 is a schematic diagram of the dressing interface of FIG. 12, illustrating additional details that may be associated with some embodiments of the therapy system of FIG. 1;

FIG. 14 is a perspective view of a conduit system that may be associated with some embodiments of the dressing interface of FIG. 3;

FIG. 15 is a cross-sectional view of the conduit system of FIG. 13 taken along line 15-15 of FIG. 14, illustrating additional details that may be associated with some embodiments;

FIG. 16 is a schematic diagrams of a slip ring that may be associated with a dressing interface;

FIG. 17 is a cross-sectional view of the slip ring of FIG. 15 taken along line 17-17 of FIG. 16, illustrating additional details that may be associated with some embodiments; and

FIG. 18 is a schematic diagram of the slip ring of FIG. 15, illustrating additional details that may be associated with some embodiments.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

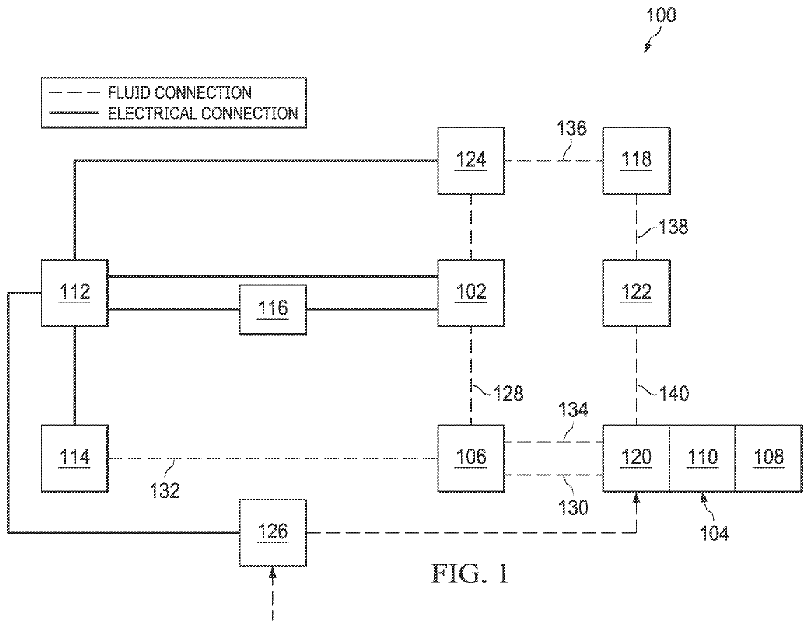

FIG. 1 is a simplified functional block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 102, and one or more distribution components. A distribution component is preferably detachable and may be disposable, reusable, or recyclable. A dressing, such as a dressing 104, and a fluid container, such as a container 106, are examples of distribution components that may be associated with some examples of the therapy system 100. A dressing may include a cover, a tissue interface, or both in some embodiments. As illustrated in the example of FIG. 1, the dressing 104 may comprise or consist essentially of a tissue interface 108, a cover 110, and one or more dressing interfaces, such as a dressing interface 120.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 104. For example, such a dressing interface may be a SENSAT.R.A.C.™ Pad available from Kinetic Concepts, Inc. of San Antonio, Texas.

The therapy system 100 may also include a regulator or controller, such as a controller 112. Additionally, the therapy system 100 may include sensors to measure operating parameters and provide feedback signals to the controller 112 indicative of the operating parameters. As illustrated in FIG. 1, for example, the therapy system 100 may include a first sensor 114 and a second sensor 116 coupled to the controller 112.

The therapy system 100 may also include a source of instillation solution, such as a solution source 118. An instillation pump 124 may be coupled to the solution source 118, as illustrated in the example embodiment of FIG. 1. The instillation pump 124 may also be fluidly coupled to the negative-pressure source 102. In some embodiments, the instillation pump 124 may be directly coupled to the negative-pressure source 102. In other embodiments, the instillation pump 124 may be indirectly coupled to the negative-pressure source 102 through other distribution components. For example, the instillation pump 124 may be fluidly coupled to the negative-pressure source 102 through the dressing 104.

A regulator, such as an instillation regulator 122, may also be fluidly coupled to the solution source 118 and the dressing 104 to ensure proper dosage of instillation solution (e.g. saline) to a tissue site. For example, the instillation regulator 122 may comprise a piston that can be pneumatically actuated by the negative-pressure source 102 to draw instillation solution from the solution source during a negative-pressure interval and to instill the solution to a dressing during a venting interval. Additionally or alternatively, the controller 112 may be coupled to the negative-pressure source 102 to control dosage of instillation solution to a tissue site. In some embodiments, the instillation regulator 122 may also be fluidly coupled to the negative-pressure source 102 through the dressing 104.

In some embodiments, components may also be coupled by virtue of physical proximity, being integral to a single structure, or being formed from the same piece of material. Coupling may also include mechanical, thermal, electrical, or chemical coupling (such as a chemical bond) in some contexts. For example, a tube may mechanically and fluidly couple the dressing 104 to the container 106. In general, components of the therapy system 100 may be coupled directly or indirectly. For example, the negative-pressure source 102 may be directly coupled to the container 106 and may be indirectly coupled to the dressing 104 through the container 106 by a conduit 128 and a negative-pressure delivery conduit 130. The negative-pressure source 102 may be electrically coupled to the controller 112 and may be fluidly coupled to one or more distribution components to provide a fluid path to a tissue site. The first sensor 114 may be fluidly coupled to the dressing 104 directly or indirectly by a conduit 132 and a pressure sensing conduit 134. Additionally, the instillation pump 124 may be coupled indirectly to the dressing interface 120 through the solution source 118 and the instillation regulator 122 by a conduit 136, a conduit 138, and an instillation delivery conduit 140.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies a position in a fluid path relatively closer to a source of negative pressure or further away from a source of positive pressure. Conversely, the term “upstream” implies a position in a fluid path relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

A negative-pressure supply, such as the negative-pressure source 102, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 102 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The tissue interface 108 can be generally adapted to partially or fully contact a tissue site. The tissue interface 108 may take many forms, and may have many sizes, shapes, or thicknesses, depending on a variety of factors, such as the type of treatment being implemented or the nature and size of a tissue site. For example, the size and shape of the tissue interface 108 may be adapted to the contours of deep and irregular shaped tissue sites. Any or all of the surfaces of the tissue interface 108 may have an uneven, coarse, or jagged profile.

In some embodiments, the tissue interface 108 may comprise or consist essentially of a manifold. A manifold in this context may comprise or consist essentially of a means for collecting or distributing fluid across the tissue interface 108 under pressure. For example, a manifold may be adapted to receive negative pressure from a source and distribute negative pressure through multiple apertures across the tissue interface 108, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 108 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 108 may also vary according to needs of a prescribed therapy. For example, the tensile strength of foam may be increased for instillation of topical treatment solutions. The 25% compression load deflection of the tissue interface 108 may be at least 0.35 pounds per square inch, and the 65% compression load deflection may be at least 0.43 pounds per square inch. In some embodiments, the tensile strength of the tissue interface 108 may be at least 10 pounds per square inch. The tissue interface 108 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 108 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 108 may also vary according to needs of a prescribed therapy. For example, the thickness of the tissue interface may be decreased to reduce tension on peripheral tissue. The thickness of the tissue interface 108 can also affect the conformability of the tissue interface 108. In some embodiments, a thickness in a range of about 5 millimeters to 10 millimeters may be suitable. In other embodiments, the tissue interface 108 can have a thickness of up to about 32 mm.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

The tissue interface 108 may further promote granulation at a tissue site when pressure within the sealed therapeutic environment is reduced. For example, any or all of the surfaces of the tissue interface 108 may have an uneven, coarse, or jagged profile that can induce microstrains and stresses at a tissue site if negative pressure is applied through the tissue interface 108.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 110 may provide a bacterial barrier and protection from physical trauma. The cover 110 may also be constructed from a material that can reduce evaporative losses and provide a fluid seal between two components or two environments, such as between a therapeutic environment and a local external environment. The cover 110 may comprise or consist of, for example, an elastomeric film or membrane that can provide a seal adequate to maintain a negative pressure at a tissue site for a given negative-pressure source. The cover 110 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 110 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 110 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and Inspire 2301 and Inspire 2327 polyurethane films, commercially available from Expopack Advanced Coatings, Wrexham, United Kingdom. In some embodiments, the cover 110 may comprise INSPIRE 2301 having an MVTR (upright cup technique) of 2600 g/m²/24 hours and a thickness of about 30 microns.

An attachment device may be used to attach the cover 110 to an attachment surface, such as undamaged epidermis, a gasket, or another cover. The attachment device may take many forms. For example, an attachment device may be a medically-acceptable, pressure-sensitive adhesive configured to bond the cover 110 to epidermis around a tissue site. In some embodiments, for example, some or all of the cover 110 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In some embodiments, a dressing interface, such as the dressing interface 120, may facilitate coupling the negative-pressure source 102 to the dressing 104. The negative pressure provided by the negative-pressure source 102 may be delivered through the negative-pressure delivery conduit 130 to a negative-pressure connector (not shown) having a first end adapted to be positioned in fluid communication with the dressing interface 120 and a second end adapted to be fluidly coupled to the negative-pressure delivery conduit 130. In some embodiments, a connector, such as the negative-pressure connector or a negative-pressure interface, may have a substantially low profile to reduce pressure points exerted on a patient by the connector. In some embodiments, the connector may be substantially rigid. In yet another example embodiment, the connector may be semi-rigid such as, for example, a T.R.A.C.® Pad or Sensa T.R.A.C.® Pad available from KCl of San Antonio, Texas. The dressing interface 120, in conjunction with the connector and the negative-pressure delivery conduit 130, delivers negative pressure within an interior portion of the cover 110 and the tissue interface 108.

A controller, such as the controller 112, may be a microprocessor or computer programmed to operate one or more components of the therapy system 100, such as the negative-pressure source 102. In some embodiments, for example, the controller 112 may be a microcontroller, which generally comprises an integrated circuit containing a processor core and a memory programmed to directly or indirectly control one or more operating parameters of the therapy system 100. Operating parameters may include the power applied to the negative-pressure source 102, the pressure generated by the negative-pressure source 102, or the pressure distributed to the tissue interface 108, for example. The controller 112 is also preferably configured to receive one or more input signals, such as a feedback signal, and programmed to modify one or more operating parameters based on the input signals.

In some embodiments, the controller 112 may receive and process data from one or more sensors, such as the first sensor 114. The controller 112 may also control the operation of one or more components of the therapy system 100 to manage the pressure delivered to the tissue interface 108. In some embodiments, controller 112 may include an input for receiving a desired target pressure and may be programmed for processing data relating to the setting and inputting of the target pressure to be applied to the tissue interface 108. In some example embodiments, the target pressure may be a fixed pressure value set by an operator as the target negative pressure desired for therapy at a tissue site and then provided as input to the controller 112. The target pressure may vary from tissue site to tissue site based on the type of tissue forming a tissue site, the type of injury or wound (if any), the medical condition of the patient, and the preference of the attending physician. After selecting a desired target pressure, the controller 112 can operate the negative-pressure source 102 in one or more control modes based on the target pressure and may receive feedback from one or more sensors to maintain the target pressure at the tissue interface 108.

Sensors, such as the first sensor 114 and the second sensor 116, are generally known in the art as any apparatus operable to detect or measure a physical phenomenon or property, and generally provide a signal indicative of the phenomenon or property that is detected or measured. For example, the first sensor 114 and the second sensor 116 may be configured to measure one or more operating parameters of the therapy system 100. In some embodiments, the first sensor 114 may be a transducer configured to measure pressure in a pneumatic pathway and convert the measurement to a signal indicative of the pressure measured. In some embodiments, for example, the first sensor 114 may be a piezo-resistive strain gauge. The second sensor 116 may optionally measure operating parameters of the negative-pressure source 102, such as a voltage or current, in some embodiments. Preferably, the signals from the first sensor 114 and the second sensor 116 are suitable as an input signal to the controller 112, but some signal conditioning may be appropriate in some embodiments. For example, the signal may need to be filtered or amplified before it can be processed by the controller 112. Typically, the signal is an electrical signal, but may be represented in other forms, such as an optical signal.

The solution source 118 may be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions. Examples of therapeutic solutions that may be suitable for some prescriptions include hypochlorite-based solutions, silver nitrate (0.5%), sulfur-based solutions, biguanides, cationic solutions, and isotonic solutions. In one illustrative embodiment, the solution source 118 may include a storage component for the solution and a separate cassette for holding the storage component and delivering the solution to the tissue site, such as a V.A.C. VeraLink™ Cassette available from Kinetic Concepts, Inc. of San Antonio, Texas.

The container 106 may be representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy. In some embodiments, the container 106 may comprise a canister having a collection chamber, a first inlet fluidly coupled to the collection chamber and a first outlet fluidly coupled to the collection chamber and adapted to receive negative pressure from a source of negative pressure. In some embodiments, a first fluid conductor may comprise a first member such as, for example, the negative-pressure delivery conduit 130 fluidly coupled between the first inlet and the tissue interface 108 by the dressing interface 120, and a second member such as, for example, the conduit 128 fluidly coupled between the first outlet and a source of negative pressure whereby the first conductor is adapted to provide negative pressure within the collection chamber to the tissue site.

The therapy system 100 may also comprise a flow regulator such as, for example, a regulator 126 fluidly coupled to a source of ambient air to provide a controlled or managed flow of ambient air to the sealed therapeutic environment provided by the dressing 104 and ultimately the tissue site. In some embodiments, the regulator 126 may control the flow of ambient fluid to purge fluids and exudates from the sealed therapeutic environment. In some embodiments, the regulator 126 may be fluidly coupled to the tissue interface 108 through the dressing interface 120. The regulator 126 may be configured to fluidly couple the tissue interface 108 to a source of ambient air. In some embodiments, the regulator 126 may be disposed within the therapy system 100 rather than being proximate to the dressing 104 so that the air flowing through the regulator 126 is less susceptible to accidental blockage during use. In some embodiments, the regulator 126 may be positioned proximate the container 106 and/or proximate a source of ambient air, where the regulator 126 is less likely to be blocked during usage.

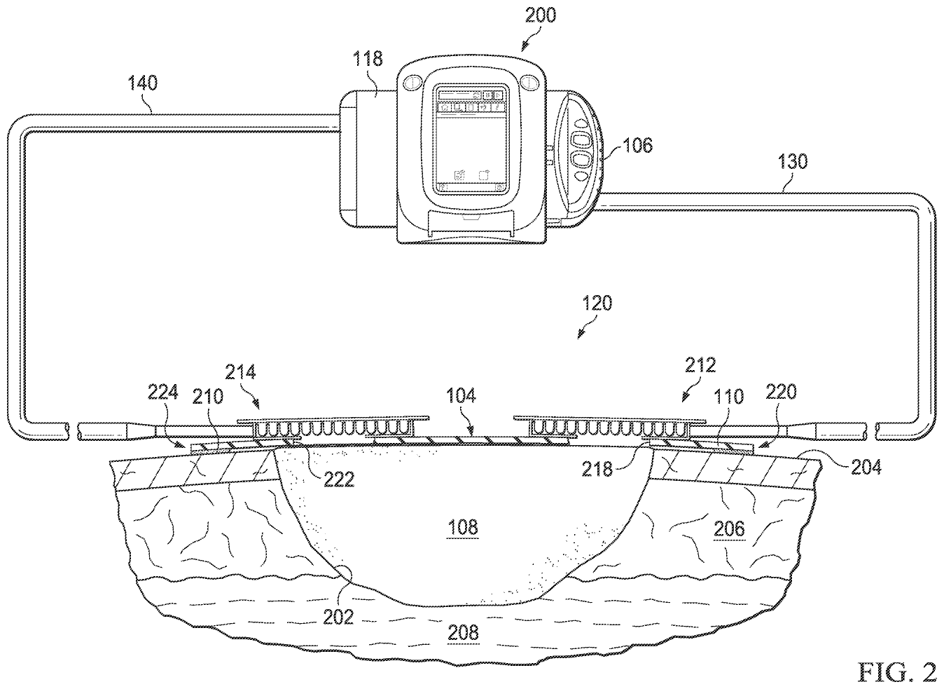

FIG. 2 is a schematic diagram of an example embodiment of the therapy system of FIG. 1, illustrating additional details that may be associated with some embodiments. Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the controller 112, the solution source 118, and other components into a therapy unit, such as a therapy unit 200. The therapy unit 200 may be, for example, a V.A.C.ULTA™ Therapy Unit available from Kinetic Concepts, Inc. of San Antonio, Texas.

In operation, the tissue interface 108 may be placed within, over, on, or otherwise proximate to a tissue site, such as a tissue site 202. If the tissue site 202 is a wound, for example, the tissue interface 108 may partially or completely fill the wound, or it may be placed over the wound. In the example of FIG. 2, the tissue site 202 extends through an epidermis 204, or generally the skin, and a dermis 206 and reaching into a hypodermis, or a subcutaneous tissue 208. The therapy system 100 may be used to treat a wound of any depth, as well as many different types of wounds, including open wounds, incisions, or other tissue sites. Treatment of the tissue site 202 may include removal of fluids originating from the tissue site 202, such as exudates or ascites, or fluids instilled into the dressing to cleanse or treat the tissue site 202, such as antimicrobial solutions.

The cover 110 may be placed over the tissue interface 108 and an attachment device 210 can seal the cover 110 to an attachment surface near the tissue site 202. For example, the cover 110 may be sealed to undamaged epidermis peripheral to the tissue site 202. Thus, the dressing 104 can provide a sealed therapeutic environment proximate to the tissue site 202, substantially isolated from the external environment, and the therapy unit 200 can reduce pressure in the sealed therapeutic environment. Negative pressure applied across the tissue site 202 through the tissue interface 108 in the sealed therapeutic environment can induce macro-strain and micro-strain in the tissue site 202, as well as remove exudates and other fluids from the tissue site 202, which can be collected in the container 106. In some embodiments, the cover may have a first end 220 and a second end 224. The first end 220 and the second end 224 may be spaced from each other on the cover 110. In some embodiments, the first end 220 and the second end 224 may represent opposite positions of a maximum extent of the cover 110. In other embodiments, the first end 220 and the second end 224 may be proximate to each other. The cover 110 may also have one or more openings, apertures, or holes. For example, the cover may have a first hole 218 disposed in the first end 220 and a second hole 222 disposed in the second end 224. In some embodiments, the first hole 218 and the second hole 222 may be positioned to maximize the distance between the first hole 218 and the second hole 222 while still being capable of being fluidly coupled to the tissue interface 108. In other embodiments, the first hole 218 and the second hole 222 may be proximate to or adjacent to each other. Each of the first hole 218 and the second hole 222 may have an effective diameter. The effective diameter of an object is a diameter of a circle having the same area as the object. For example, a square having a side of 2 mm may have an effective diameter of 2.25 mm.

The dressing interface 120 may be coupled to the cover 110 to fluidly couple the therapy unit 200 to the tissue interface 108. In some embodiments, the therapy unit 200 may be fluidly coupled to two different locations on the same dressing 104 by the dressing interface 120. For example, the negative-pressure source 102 may be fluidly coupled to the tissue interface 108 at a first location, while the solution source 118 may be fluidly coupled to the tissue interface 108 at a second location. In some embodiments, the dressing interface 120 may include more than one fluid coupling. For example, the dressing interface 120 may comprise a first bridge 212 and a second bridge 214. The first bridge 212 may fluidly couple the therapy unit 200 to the dressing 104 via the negative-pressure delivery conduit 130. The second bridge 214 may fluidly couple the therapy unit 200 to the dressing 104 via the instillation delivery conduit 140. In some embodiments, the first bridge 212 may be coupled to the cover 110 adjacent to the first hole 218, and the second bridge 214 may be coupled to the cover 110 adjacent to the second hole 222. The first bridge 212 and the second bridge 214 may be adapted to be in fluid communication with the tissue interface 108 via the first hole 218 and the second hole 222, respectively. Generally, the first bridge 212 and the second bridge 214 may be substantially flat and flexible, but also compressible without occluding or blocking the fluid pathway between the negative-pressure delivery conduit 130 and the tissue interface 108 or the fluid pathway between the instillation delivery conduit 140 and the tissue interface 108.

FIG. 3 is a schematic diagram illustrating additional details that may be associated with some example embodiments of the dressing interface 120. The first bridge 212 and the second bridge 214 may be configured to be independently fluidly coupled to the tissue interface 108. In some embodiments, the first bridge 212 and the second bridge 214 may have a length extending from a first end to a second end. For example, the first bridge 212 may have a first end 301 and a second end 302. The first end 301 may be configured to be fluidly coupled to the tissue interface 108, and the second end 302 may be configured to be fluidly coupled to at least one conduit, such as a first bridge conduit 304. In some embodiments, the first bridge 212 may comprise an applicator portion, such as a first applicator 306, positioned at the first end 301 and a first elongate member 307 extending from the first applicator 306 to the second end 302. In some embodiments, the first applicator 306 and the first elongate member 307 may be integral. In other embodiments, the first applicator 306 and the first elongate member 307 may be formed as separate components that are coupled together to form the first bridge 212. The first applicator 306 may fluidly couple the first bridge 212 to the tissue interface 108. The first applicator 306 may be configured to be fluidly coupled to the tissue interface 108 via the first hole 218 in the cover 110. The first applicator 306 may be a circular, oval, elliptical, or other rounded shape suitable for applying therapy to the tissue interface 108, depending on the size and nature of the tissue interface 108. In other embodiments, the first applicator 306 may have a polygonal, square, rectangular, triangular, or amorphous shape. The first applicator 306 may have an effective diameter greater than the effective diameter of the first hole 218.

The first elongate member 307 can comprise a generally rectangular body having a length greater than its width. In some embodiments, the length of the first elongate member 307 may be determined by the desired treatment. For example, the length of the first elongate member 307 may be longer for treating a patient's foot than for treating a patient's knee. In some embodiments, the length of the first elongate member 307 may be between about 20 cm and about 60 cm. In some embodiments, the length of the first elongate member 307 may be ten times the width of the first elongate member 307.

In some embodiments, the width of the first elongate member 307 may be determined by the amount of fluid to be removed from the tissue site 202. For example, the width of the first elongate member 307 may be smaller when removing lower volumes of fluid having a low viscosity. In other embodiments, the width of the first elongate member 307 may need to be greater to remove a higher volume of fluid having a higher viscosity. In some embodiments, the width of the first elongate member 307 may be between about 1 cm and about 5 cm. In some embodiments, the width of the first elongate member 307 may be less than the effective diameter of the first applicator 306. In other embodiments, the width of the first elongate member 307 may be equal to or greater than the effective diameter of the first applicator 306. A center of the width of the first elongate member 307 may be aligned with a diameter of the first applicator 306. In other embodiments, the center of the width of the first elongate member 307 may be offset from the diameter of the first applicator 306.

In some embodiments, the first bridge 212 may comprise a first fluid pathway 308 extending from the first end 301 to the second end 302. The first fluid pathway 308 may comprise a plurality of features, such as flexible projections, flexible standoffs, or closed cells, projecting into the first fluid pathway 308. For example, the first bridge 212 may comprise a first plurality of features 310 disposed throughout and along the length of the first bridge 212 projecting into the first fluid pathway 308. In some embodiments, the first plurality of features 310 may be positioned in the first elongate member 307 and the first applicator 306 of the first bridge 212. In other embodiments, the first plurality of features 310 may be positioned only in the first elongate member 307 of the first bridge 212. In some embodiments, the first plurality of features 310 may have a volumetric shape that is any one of a hemispherical, conical, cylindrical, square, rectangular, or geodesic shape.

In some embodiments, the second bridge 214 may have a length extending from a first end 312 and a second end 313. The first end 312 may be configured to be fluidly coupled to the tissue interface 108, and the second end 313 may be configured to be fluidly coupled to at least two conduits, such as a second bridge conduit 314 and an instillation conduit 316. In other embodiments, the second end 313 may be fluidly coupled to a single conduit, such as a multi-lumen conduit. In some embodiments, the second bridge 214 may comprise an applicator portion, such as a second applicator 318, positioned at the first end 312 and a second elongate member 319 extending from the second applicator 318 to the second end 313. In some embodiments, the second applicator 318 and the second elongate member 319 may be integral. In other embodiments, the second applicator 318 and the second elongate member 319 may be formed as separate components that are coupled together to form the second bridge 214. The second applicator 318 may fluidly couple the second bridge 214 to the tissue interface 108. The second applicator 318 may be configured to be fluidly coupled to the tissue interface 108 via the second hole 222 in the cover 110. The second applicator 318 may be a circular, oval, elliptical, or other rounded shape suitable for applying therapy to the tissue interface 108, depending on the size and nature of the tissue interface 108. In other embodiments, the second applicator 318 may have a polygonal, square, rectangular, triangular, or amorphous shape. The second applicator 318 may have an effective diameter greater than the effective diameter of the second hole 222.

The second elongate member 319 can comprise a generally rectangular body having a length greater than its width. In some embodiments, the length of the second elongate member 319 may be determined by the desired treatment. For example, the length of the second elongate member 319 may be longer for treating a patient's foot than for treating a patient's knee. In some embodiments, the length of the second elongate member 319 may be between about 20 cm and about 60 cm. In some embodiments, the length of the second elongate member 319 may be ten times the width of the second elongate member 319.

In some embodiments, the width of the second elongate member 319 may be determined by the amount of fluid to be removed from the tissue site 202. For example, the width of the second elongate member 319 may be smaller when removing lower volumes of fluid having a low viscosity. In other embodiments, the width of the second elongate member 319 may need to be greater to remove a higher volume of fluid having a higher viscosity. In some embodiments, the width of the second elongate member 319 may be between about 1 cm and about 5 cm. In some embodiments, the width of the second elongate member 319 may be less than the effective diameter of the second applicator 318. In other embodiments, the width of the second elongate member 319 may be equal to or greater than the effective diameter of the second applicator 318. A center of the width of the second elongate member 319 may be aligned with a diameter of the second applicator 318. In other embodiments, the center of the width of the second elongate member 319 may be offset from the diameter of the second applicator 318.

In some embodiments, the second bridge 214 may comprise at least two barriers or walls, such as a first barrier 326 and a second barrier 328. The first barrier 326 and the second barrier 328 may be disposed in the second elongate member 319 and extend from the first end 312 into the second applicator 318. In some embodiments, the first barrier 326 and the second barrier 328 may extend to the second end 313.

In some embodiments, the second bridge 214 may comprise a second fluid pathway 320, a third fluid pathway 322, and a fourth fluid pathway 324, each extending from the first end 312 toward the second end 313. The second fluid pathway 320 may be formed between the first barrier 326 and the second barrier 328. The third fluid pathway 322 and the fourth fluid pathway 324 may be formed outbound of the second fluid pathway 320. For example, the third fluid pathway 322 may be disposed between the first barrier 326 and an exterior edge of the second elongate member 319, and the fourth fluid pathway 324 may be disposed between the second barrier 328 and an opposing exterior edge of the second elongate member 319. In some embodiments, each of the second fluid pathway 320, the third fluid pathway 322, and the fourth fluid pathway 324 may extend through the second elongate member 319 and into the second applicator 318. In other embodiments, the second fluid pathway 320, the third fluid pathway 322, and the fourth fluid pathway 324 may extend through the second elongate member 319 up to the second applicator 318. In some embodiments, the second fluid pathway 320, the third fluid pathway 322, and the fourth fluid pathway 324 may be fluidly coupled to each other in the second applicator 318. In other embodiments, the second fluid pathway 320, the third fluid pathway 322, and the fourth fluid pathway 324 may be fluidly isolated from each other in the second applicator 318.

In some embodiments, the second fluid pathway 320, the third fluid pathway 322, and a fourth fluid pathway 324 may comprise a plurality of features, such as flexible projections, flexible standoffs, or closed cells projecting into the second fluid pathway 320, the third fluid pathway 322, and a fourth fluid pathway 324. The plurality of features may be disposed throughout and along the length of the second bridge 214. For example, the second bridge 214 may comprise a second plurality of features 330 projecting into the second fluid pathway 320, a third plurality of features 332 projecting into the third fluid pathway 322, and a fourth plurality of features 334 projecting into the fourth fluid pathway 324. In some embodiments, the second plurality of features 330, the third plurality of features 332, and the fourth plurality of features 334 may be positioned only in the second elongate member 319 of the second bridge 214. In some embodiments, the second plurality of features 330, the third plurality of features 332, and the fourth plurality of features 334 may be positioned in the second elongate member 319 and a portion of the second applicator 318. In other embodiments, the second plurality of features 330, the third plurality of features 332, and the fourth plurality of features 334 may be positioned in the second elongate member 319 and the second applicator 318 of the second bridge 214. In some embodiments, the second plurality of features 330, the third plurality of features 332, and the fourth plurality of features 334 may have a volumetric shape that is any one of a hemispherical, conical, cylindrical, square, rectangular, or geodesic shape.

In some embodiments, the first bridge conduit 304 may be fluid coupled to the first bridge 212 via the use of a connector, such as the negative-pressure connector or the negative-pressure interface. For example, the negative-pressure connector can be coupled to the second end 302 of the first bridge 212, providing a fluid path from the first fluid pathway 308 to an environment external to the first bridge 212. The first bridge conduit 304 can be coupled to the negative-pressure connector, and the negative-pressure connector can fluidly couple a lumen of the first bridge conduit 304 to the first fluid pathway 308. In other embodiments, the first fluid pathway 308 may be directly coupled to the first bridge conduit 304. For example, the first bridge conduit 304 may be inserted into and sealed to the second end 302 of the first bridge 212. In the example of FIG. 3, the first bridge conduit 304 is directly welded to the first bridge 212 and fluidly coupled to the first fluid pathway 308.

In some embodiments, the second bridge conduit 314 and the instillation conduit 316 may be fluidly coupled to the second bridge 214 via the use of an interface pad, such as an interface 342. In the example of FIG. 3, the interface 342 fluidly couples the second bridge conduit 314 and the instillation conduit 316 to the second end 313 of the second bridge 214. In some embodiments, the interface 342 may fluidly couple the second fluid pathway 320 to the instillation conduit 316 and may fluidly couple both the third fluid pathway 322 and the fourth fluid pathway 324 to the second bridge conduit 314. In some embodiments, the second bridge conduit 314 may be a multi-lumen conduit and the interface 342 may split the multi-lumen conduit into the required pathways for fluid coupling to the third fluid pathway 322 and the fourth fluid pathway 324. In other embodiments, the second bridge conduit 314 may be a multi-lumen conduit directly coupled to the second end 313 of the second bridge 214. In such embodiments, at least one lumen of the multi-lumen conduit may be coupled to the third fluid pathway 322 and at least one other lumen of the multi-lumen conduit may be coupled to the fourth fluid pathway 324. In still other embodiments, the second fluid pathway 320, the third fluid pathway 322, and the fourth fluid pathway 324 may be directly coupled to their own respective conduits at the second end 313 of the second bridge 214. Additionally or alternatively, the second bridge conduit 314, and the instillation conduit 316 may be directly welded within the second bridge 214.

A connector, such as a connector 336, may be configured to fluidly couple the first bridge 212 and the second bridge 214 to the therapy unit 200. In some embodiments, the connector 336 may be fluidly coupled to the first bridge 212 via the first bridge conduit 304. The connector 336 may be fluidly coupled to the second bridge 214 via the second bridge conduit 314 and the instillation conduit 316. In some embodiments, the connector 336 may be configured to merge the first bridge conduit 304, the instillation conduit 316, and the second bridge conduit 314 into a unibody object having a plurality of independent lumens for fluid coupling to the therapy unit 200.

In some embodiments, the connector 336 may be fluidly coupled to the therapy unit 200 via the instillation delivery conduit 140 and a multi-lumen conduit 338. The connector 336 may fluidly couple the first bridge conduit 304 and the second bridge conduit 314 to the multi-lumen conduit 338. In some embodiments, the multi-lumen conduit 338 may comprise the negative-pressure delivery conduit 130 and the pressure sensing conduit 134. The connector 336 may fluidly couple the first bridge conduit 304 to the negative-pressure delivery conduit 130 within the multi-lumen conduit 338, and may fluidly couple the second bridge conduit 314 to the pressure sensing conduit 134 within the multi-lumen conduit 338. In some embodiments, the multi-lumen conduit 338 may be fluidly coupled to the negative-pressure source 102 contained within therapy unit 200 to create a negative-pressure pathway. The multi-lumen conduit 338 may also be fluidly coupled to a pressure sensor, such as the first sensor 114, contained within therapy unit 200 to create a sensing pathway. In some embodiments, the connector 336 may also fluidly couple the instillation conduit 316 to the instillation delivery conduit 140. The instillation delivery conduit 140 may be fluidly coupled to the solution source 118 contained within the therapy unit 200 to create an instillation pathway from the therapy unit 200 to the second bridge 214. In other embodiments, the instillation delivery conduit 140 may pass through the connector 336 for direct fluid coupling to the second bridge 214.

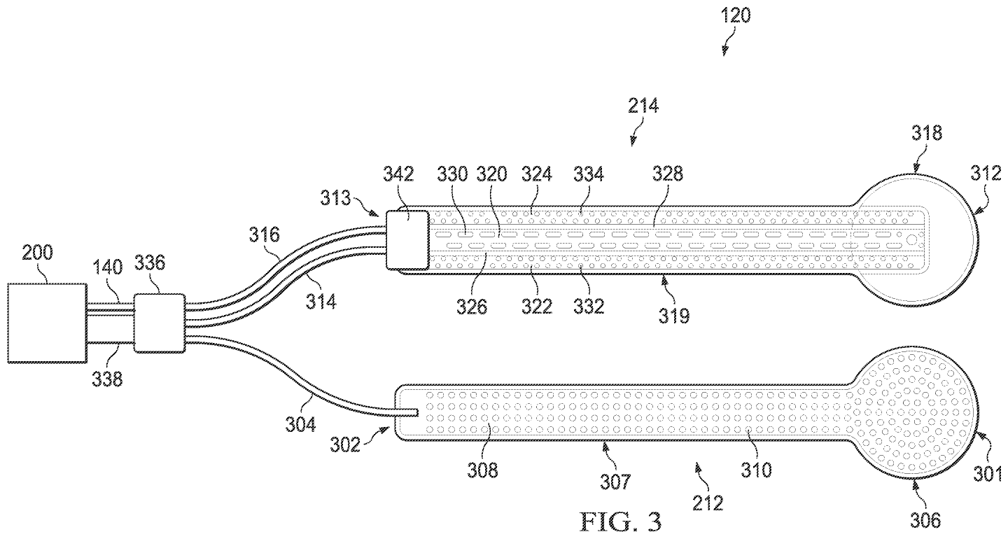

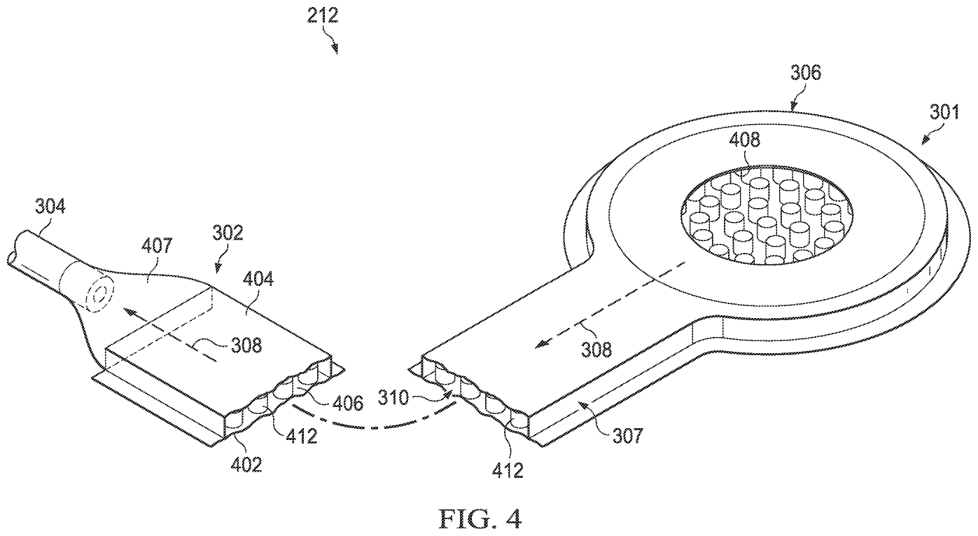

FIG. 4 is a segmented perspective view of the bottom of the first bridge 212, illustrating additional details that may be associated with some embodiments. In some embodiments, the first bridge 212 may comprise a top layer such as, for example, a first layer 402, and a base layer such as, for example, a second layer 404. The first layer 402 may be coupled to the second layer 404 around a periphery of the first layer 402 to form a sealed space, such as a first sealed space 406, between the first layer 402 and the second layer 404 of the first bridge 212. The second layer 404 may have an inner surface facing the first sealed space 406 and an outer surface facing away from the first sealed space 406. In some embodiments, the outer surface of the second layer 404 may be configured to contact the tissue interface 108, the cover 110, and at least a portion of undamaged epidermis peripheral to the tissue site 202. The first layer 402 may also have an inner surface facing the first sealed space 406 and an outer surface facing away from the first sealed space 406. In some embodiments, the outer surface of the first layer 402 may be configured to be exposed to the ambient environment. In some embodiments, the first layer 402 may include the first plurality of features 310 disposed along a length of the first bridge 212. The first plurality of features 310 may extend from the inner surface of the first layer 402 into the first sealed space 406. The second layer 404 may cover the first plurality of features 310. The first fluid pathway 308 may be formed within the first sealed space 406 between the first layer 402, the second layer 404, and the first plurality of features 310.

In some embodiments, the first bridge 212 may include an interface 407 fluidly coupled to the first bridge 212 and extending from the second end 302. The first bridge 212 and the interface 407 may have a substantially flat profile and the interface 407 may be configured to fluidly couple the first fluid pathway 308 to a tube or conduit, such as the first bridge conduit 304. In some embodiments, the first bridge conduit 304 may comprise a single lumen for delivering negative pressure to the first bridge 212.

The first bridge 212 may also include an aperture 408 disposed in the second layer 404 at the first end 301. For example, the aperture 408 may be disposed in the second layer 404 of the first applicator 306. In some embodiments, a portion of the first sealed space 406 within the first applicator 306 may be exposed by the aperture 408. The aperture 408 may be configured to fluidly couple the first sealed space 406 to another device or object. For example, the aperture 408 may fluidly couple the first fluid pathway 308 to the tissue interface 108. In other embodiments, the first applicator 306 may comprise an adhesive on the outer surface of the second layer 404. The adhesive may be used to adhere the first bridge 212 to the cover 110 of the dressing 104.

The first plurality of features 310 may comprise flexible projections, flexible standoffs, or closed cells, such as closed cells 412. Each of the closed cells 412 may have a bottom portion extending from the inner surface of the first layer 402 and a top portion extending into the first sealed space 406 toward the second layer 404. Within the first applicator 306, the top portion of the closed cells 412 may extend from the first layer 402 toward the aperture 408. In some embodiments, the top portion of the closed cells 412 may come in contact with the second layer 404. In other embodiments, the top portion of the closed cells 412 may be coupled to the second layer 404. In some embodiments, the plurality of closed cells 412 of the first bridge 212 may have a volumetric shape. For example, the closed cells 412 may have a shape that is any one of a hemispherical, conical, cylindrical, square, rectangular, or geodesic shape. In some example embodiments the first plurality of features 310 may further comprise projections or nodes (not shown) positioned on the top portion of the closed cells 412.

In other embodiments, the closed cells 412 may only be disposed in the first applicator 306 and the remaining portion of the first bridge 212 may contain a fabric material instead of the closed cells 412. For example, the first fluid pathway 308 may comprise a manifolding layer disposed between the first layer 402 and the second layer 404 of the first bridge 212. In some embodiments, the manifolding layer may include one or more of a reticulated foam, combinations of foam and fabric (such as various textiles manufactured by Milliken & Company), a coated or treated foam (such as plasma treated), a woven layer, a felted reticulated foam, or a 3D spacer fabric. Additionally or alternatively, the manifolding layer may comprise or consist essentially of a low-profile 3D polyester textile, such as textiles manufactured by Baltex. In some embodiments, the manifolding layer may have a thickness between about 3 millimeters to about 8 millimeters.

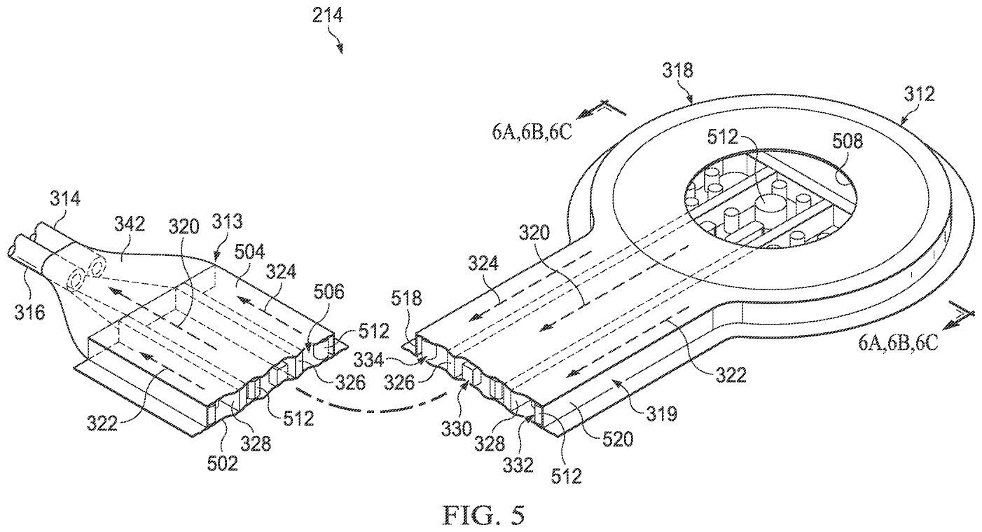

FIG. 5 is a segmented perspective view of the bottom of the second bridge 214 of the dressing interface 120 of FIG. 3. The second bridge 214 may comprise a first layer 502 and a second layer 504. The first layer 502 may be coupled to the second layer 504 around a periphery of the first layer 502 to form a sealed space, such as a second sealed space 506, within the second bridge 214. The second layer 504 may have an inner surface facing the second sealed space 506 and an outer surface facing away from the second sealed space 506. In some embodiments, the outer surface of the second layer 504 may be configured to contact the tissue interface 108, the cover 110, and at least a portion of undamaged epidermis peripheral to the tissue site 202. The first layer 502 may also have an inner surface facing the second sealed space 506 and an outer surface facing away from the second sealed space 506. In some embodiments, the outer surface of the first layer 502 may be configured to be exposed to the ambient environment. In some embodiments, the first layer 502 may include the second plurality of features 330, the third plurality of features 332, and the fourth plurality of features 334 disposed along a length of the second bridge 214. The second plurality of features 330, the third plurality of features 332, and the fourth plurality of features 334 may extend from the inner surface of the first layer 502 into the second sealed space 506. The second layer 504 may cover the second plurality of features 330, the third plurality of features 332, and the fourth plurality of features 334. The second fluid pathway 320 may be formed within the second sealed space 506 between the first layer 502, the second layer 504, and the second plurality of features 330; the third fluid pathway 322 may be formed within the second sealed space 506 between the first layer 502, the second layer 504, and the third plurality of features 332; and the fourth fluid pathway 324 may be formed within the second sealed space 506 between the first layer 502, the second layer 504, and the fourth plurality of features 334.

In some embodiments, the second fluid pathway 320, the third fluid pathway 322, and the fourth fluid pathway 324 may be formed side-by-side within the second sealed space 506 by sealing between each pathway. In some embodiments, the first barrier 326 and the second barrier 328 may divide the second sealed space 506 into at least three sealed spaces or isolated fluid pathways between the first layer 502 and the second layer 504 of the second bridge 214. In some embodiments, the second fluid pathway 320 may be within the second sealed space 506 between the first barrier 326 and the second barrier 328. The third fluid pathway 322 and the fourth fluid pathway 324 may be within the second sealed space 506 outbound of the second fluid pathway 320. For example, the third fluid pathway 322 may be between the first barrier 326 and a first seal, such as a first seal 518, formed between a first portion of the first layer 502 and the second layer 504. The fourth fluid pathway 324 may be formed between the second barrier 328 and a second seal, such as a second seal 520, formed between a second portion of the first layer 502 and the second layer 504. In some embodiments, the first seal 518 and the second seal 520 may be formed by welding the first layer 502 to the second layer 504. In other embodiments, the first barrier 326 and the second barrier 328 may be formed by welding the first layer 502 to the second layer 504.

In some embodiments, the second bridge 214 may include the interface 342 fluidly coupled to the second bridge 214 and extending from the second end 313. The second bridge 214 may have a substantially flat profile and the interface 342 may be configured to fluidly couple the second bridge 214 to at least one tube or conduit. For example, the interface 342 may be configured to fluidly couple the second bridge 214 to the second bridge conduit 314 and the instillation conduit 316. In some embodiments, the interface 342 may fluidly couple the second bridge conduit 314 to the third fluid pathway 322 and the fourth fluid pathway 324, forming a portion of the sensing pathway. The interface 342 may also fluidly couple the instillation conduit 316 to the second fluid pathway 320 for delivery of instillation fluid.

The second bridge 214 may also include an aperture 508 disposed in the second layer 504 at the first end 303. For example, the aperture 508 may be disposed in the second layer 504 of the second applicator 318. In some embodiments, a portion of the second sealed space 506 within the second applicator 318 may be exposed by the aperture 508. The aperture 508 may be configured to fluidly couple the second sealed space 506 to another device or object. For example, the aperture 508 may fluidly couple the second fluid pathway 320, the third fluid pathway 322, and the fourth fluid pathway 324 to the tissue interface 108. In some embodiments, the first barrier 326 and the second barrier 328 may extend only partially into the second applicator 318 so that the ends of the first barrier 326 and the second barrier 328 are exposed by the aperture 508. In such embodiments, the second fluid pathway 320, the third fluid pathway 322, and the fourth fluid pathway 324 may be in fluid communication with the second sealed space 506 of the second applicator 318 and can be adapted to be in fluid communication with the tissue interface 108. In other embodiments, the first barrier 326 and the second barrier 328 may extend beyond the aperture 508. In other embodiments, the second applicator 318 may comprise an adhesive on the outer surface of the second layer 504. The adhesive may be used to adhere the second bridge 214 to the cover 110 of the dressing 104.

Like the first bridge 212, the second plurality of features 330, the third plurality of features 332, and the third plurality of features 332 may comprise flexible projections, flexible standoffs, or closed cells, such as closed cells 512. Each of the closed cells 512 may have a bottom portion extending from the inner surface of the first layer 502 and a top portion extending into the second sealed space 506 toward the second layer 504. Within the second applicator 318, the top portion of the closed cells 512 may extend from the first layer 502 toward the aperture 508. In some example embodiments, the top portion of the closed cells 512 may come in contact with the second layer 504. In other embodiments, the top portion of the closed cells 512 may be coupled to the second layer 504. In some embodiments, the plurality of closed cells 512 of the second bridge 214 may have a volumetric shape. For example, the closed cells 512 may have a shape that is any one of a hemispherical, conical, cylindrical, square, rectangular, or geodesic shape. In some example embodiments the second plurality of features 330, the third plurality of features 332, and the third plurality of features 332 may comprise projections or nodes (not shown) positioned on the top portion of the closed cells 512.

In other embodiments, the closed cells 512 may only be disposed in the second applicator 318 and the remaining portion of the second bridge 214 may contain a fabric material instead of the closed cells 512. For example, the second fluid pathway 320, the third fluid pathway 322, and the fourth fluid pathway 324 may comprise a manifolding layer disposed between the first layer 502 and the second layer 504 of the second bridge 214. In some embodiments, the manifolding layer may include one or more of a reticulated foam, combinations of foam and fabric (such as various textiles manufactured by Milliken & Company), a coated or treated foam (such as plasma treated), a woven layer, a felted reticulated foam, or a 3D spacer fabric. Additionally or alternatively, the manifolding layer may comprise or consist essentially of a low-profile 3D polyester textile, such as textiles manufactured by Baltex. In some embodiments, the manifolding layer may have a thickness between about 3 millimeters to about 8 millimeters.

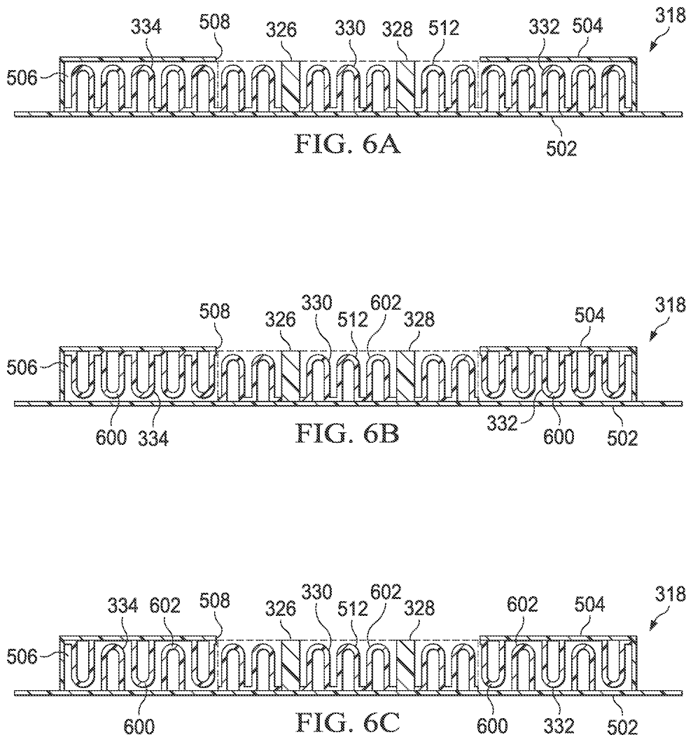

FIG. 6A shows a cross-sectional view taken of the second applicator 318 taken along line 6A-6A in FIG. 5, illustrating additional details that may be associated with some embodiments. In some embodiments, the first barrier 326 and the second barrier 328 may be positioned within the second sealed space 506 and may extend into at least a portion of the second applicator 318. If the second applicator 318 is coupled to the cover 110 and fluidly coupled to the tissue interface 108, the first barrier 326 and the second barrier 328 may divide the second sealed space 506 within the second applicator 318 into three portions comprising at least a portion of each of the second plurality of features 330, the third plurality of features 332, and the fourth plurality of features 334. In some embodiments, the second plurality of features 330, the third plurality of features 332, and the fourth plurality of features 334 may comprise the closed cells 512. As indicated above with respect to the second applicator 318 of the second bridge 214, the top portion of the closed cells 512 may extend from the first layer 502 toward the aperture 508 of the second layer 504. In some embodiments, the closed cells 512 within the second applicator 318 and exposed by the aperture 508 may be adapted to come in direct contact with the tissue interface 108. In the second sealed space 506 outside the aperture 508, the closed cells 512 may have a bottom portion extending from the first layer 502 and a top portion extending into the second sealed space 506 toward the second layer 504.

FIG. 6B shows a section view of another example of the second applicator 318 taken along line 6B-6B in FIG. 5, illustrating additional details that may be associated with some embodiments. In some embodiments, the plurality of closed cells 512 may comprise a first plurality of features, such as a first plurality of closed cells 600, and a second plurality of closed cells 602. The first plurality of closed cells 600 may have a bottom portion extending from the second layer 504 and a top portion extending into the second sealed space 506 toward the first layer 502. In some embodiments, the first plurality of closed cells 600 may be positioned outside the aperture 508. The second plurality of closed cells 602 may have a bottom portion extending from the first layer 502 and a top portion extending toward the aperture 408 of the second layer 504. In some embodiments, the second plurality of closed cells 602 may be disposed in the second applicator 318 and exposed by the aperture 508. In some embodiments, the second plurality of closed cells 602 may be adapted to come in direct contact with the tissue interface 108.

FIG. 6C shows a section view of another example embodiment of the second applicator 318 taken along line 6C-6C in FIG. 5, illustrating additional details that may be associated with some embodiments. In some embodiments, the second bridge 214 may comprise the second plurality of closed cells 602 within the second applicator 318. The second plurality of closed cells 602 within the second applicator 318 may be exposed by the aperture 508 and adapted to come in direct contact with the tissue interface 108. In some embodiments, the portions of the second sealed space 506 outside the aperture 508 may comprise both the first plurality of closed cells 600 and the second plurality of closed cells 602. The first plurality of closed cells 600 positioned within the second sealed space 506 and outside the aperture 508 may have a bottom portion extending from the second layer 504 and a top portion extending into the second sealed space 506 toward the first layer 502. The second plurality of closed cells 602 positioned within the second sealed space 506 and outside the aperture 508 may have a bottom portion extending from the first layer 502 and a top portion extending into the second sealed space 506 toward the second layer 504. In some embodiments, the closed cells 512 positioned within the second sealed space 506 and outside the aperture 508 may alternate between a closed cell from the first plurality of closed cells 600 and a closed cell from the second plurality of closed cells 602.

In other embodiments the second bridge 214 may comprise two sets of a plurality of features, one set extending from the inner surface of the first layer 502 and another set extending from the inner surface of the second layer 504. The two sets of the plurality of features may comprise two sets of closed cells 412. The two sets of closed cells 412 may be opposingly aligned so that the upper portion of the closed cells 412 extending from the first layer 502 are aligned with the upper portion of the closed cells 412 extending from the second layer 504. In some embodiments, the two sets of the plurality of features engage each other to double the height of the second bridge 214. Such a configuration may be used for all fluid pathways, such as the second fluid pathway 320, the third fluid pathway 322, and the fourth fluid pathway 324 of the second bridge 214. In some embodiments, such a configuration may only be used for fluid pathways that transmit fluid using negative pressure and not other fluid pathways. In other embodiments, the two sets of closed cells 412 may be offset from each other.

In some embodiments, the first bridge 212 may be similarly constructed as illustrated in FIGS. 6A-6C. For example, the closed cells 412 may be arranged similar to the arrangement of the closed cells 512, the first plurality of closed cells 600, and the second plurality of closed cells 602 illustrated in FIGS. 6A-6C. In some embodiments of the first bridge 212, the first barrier 326 and the second barrier 328 may be removed. Additionally or alternatively, the first bridge 212 may comprise two sets of a plurality of features, one set extending from the inner surface of the first layer 402 and another set extending from the inner surface of the second layer 404. The two sets of the plurality of features may comprise two sets of closed cells 412. The two sets of closed cells 412 may be opposingly aligned so that the upper portion of the closed cells 412 extending from the first layer 402 face are aligned with the upper portion of the closed cells 412 extending from the second layer 404. In such embodiments, the two sets of the plurality of features engage each other to double the height of the first bridge 212. In other embodiments, the two sets of closed cells 412 may be offset from each other.

In some example embodiments, the first layer 402, the first layer 502, the second layer 404, the second layer 504, the closed cells 412, and the closed cells 512 may be formed from a non-porous, polymeric film that may comprise any flexible material that can be manipulated to enclose the closed cells, including various thermoplastic materials, e.g., polyethylene homopolymer or copolymer, polypropylene homopolymer or copolymer, etc. Non-limiting examples of suitable thermoplastic polymers include polyethylene homopolymers, such as low density polyethylene (LDPE) and high density polyethylene (HDPE), and polyethylene copolymers such as, e.g., ionomers, EVA, EMA, heterogeneous (Zeigler-Natta catalyzed) ethylene/alpha-olefin copolymers, and homogeneous (metallocene, single-cite catalyzed) ethylene/alpha-olefin copolymers. Ethylene/alpha-olefin copolymers are copolymers of ethylene with one or more comonomers selected from C3 to C20 alpha-olefins, such as 1-butene, 1-pentene, 1-hexene, 1-octene, methyl pentene and the like, in which the polymer molecules comprise long chains with relatively few side chain branches, including linear low density polyethylene (LLDPE), linear medium density polyethylene (LMDPE), very low density polyethylene (VLDPE), and ultra-low density polyethylene (ULDPE). Various other materials are also suitable such as, e.g., polypropylene homopolymer or polypropylene copolymer (e.g., propylene/ethylene copolymer), polyesters, polystyrenes, polyamides, polycarbonates, etc. In some embodiments, the first layer 402, the first layer 502, the second layer 404, the second layer 504, the closed cells 412, and the closed cells 512 forming the first bridge 212 and the second bridge 214 may have a hardness in a range of about 20 Shore A to about 70 Shore A.

In some example embodiments, the first layer 402, the first layer 502, the second layer 404, the second layer 504, the closed cells 412, and the closed cells 512 may comprise a polymeric film such as, for example, a thermoplastic polyurethane (TPU) film that is permeable to water vapor but impermeable to liquid. The first layer 402, the first layer 502, the second layer 404, and the second layer 504 may be in various degrees breathable and may have MVTRs which are proportional to their thickness. For example, the MVTR may be at least 300 g/m² per twenty-four hours in some embodiments. For permeable materials, the permeability generally should be low enough to maintain a desired negative pressure for the desired negative pressure therapy treatment.

In some example embodiments, the layer having the closed cells may be formed from two sheets of polymeric film having inner surfaces coupled together to form sealed regions defining the plurality of closed cells. The two sheets of polymeric film may be a single sheet of material having two laminae or two separate sheets that are coupled together to form the closed cells. The sheets of polymeric film may initially be separate sheets that are brought into superposition and sealed or they may be formed by folding a single sheet unto itself with a heat sealable surface facing inward. Each sheet of the polymeric film also may be a monolayer or multilayer structure depending on the application or the desired structure of the closed cells. In some embodiments, the layer having the closed cells may be formed by embossing, vacuum forming, or thermoforming. Additionally or alternatively, the layer having the closed cells may be formed by processing molten or liquid solid material, such as thermoplastic polyurethane or a two-part polyurethane resin mix with a hardness in the range of 20 Shore A to about 70 Shore A using methods such as injection molding, compression molding, and dip molding.

The closed cells formed by the polymeric film may be resistant to collapsing from the negative pressure when applied to the dressing interface 120 and the tissue site 202. For example, if the first bridge 212 and the second bridge 214 are positioned at the tissue site 202 and negative pressure is applied as described above, the closed cells formed by the polymeric film are structured so that they do not completely collapse from apposition forces exerted on the first bridge 212, the second bridge 214, and the tissue site 202 from the application of negative pressure. For example, the closed cells 412, such as the first plurality of features 310, may provide a cushion to help prevent the first sealed space 406 of the first bridge 212 from collapsing as a result of external forces. Similarly, the closed cells 512, such as the second plurality of features 330, the third plurality of features 332, and the fourth plurality of features 334, may provide a cushion to help prevent the second sealed space 506 of the second bridge 214 from collapsing as a result of external forces. In one embodiment, the polymeric film possesses sufficient tensile strength to resist stretching under the apposition forces created by negative pressure wound therapy. The tensile strength of a material is the ability of material to resist stretching as represented by a stress-strain curve where stress is the force per unit area, i.e., pascals (Pa), newtons per square meter (N/m²), or pounds per square inch (psi). The ultimate tensile strength (UTS) is the maximum stress the material can withstand while being stretched before failing or breaking. Many materials display a linear elastic behavior defined by a linear stress-strain relationship often extending up to a nonlinear region represented by the yield point, i.e., the yield strength of a material. For example, high density polyethylene (HDPE) has a high tensile strength and low-density polyethylene (LDPE) has a slightly lower tensile strength, which are suitable materials for the sheets of non-porous, polymeric film as set forth above. Linear low density polyethylene (LLDPE) is often used as well because the material stretches very little as the force is increased up to the yield point of the material. Thus, the closed cells are able to resist collapsing (or stretching) when subjected to an external force or pressure. For example, HDPE has a UTS of about 37 MPa and may have a yield strength that ranges from about 26-33 MPa depending on the thickness of the material, while LDPE has somewhat lower values.

In some example embodiments, the first layer 402, the first layer 502, the second layer 404, the second layer 504, the closed cells 412, and the closed cells 512 may comprise a thermoplastic polyurethane (TPU) film as described above. The thermoplastic polyurethane film may be, for example, a Platilon® thermoplastic polyurethane film available from Convestro LLC, that may have a UTS of about 60 MPa and may have a yield strength of approximately 11 MPa or greater than about 10 MPa depending on the thickness of the material. Therefore, in some example embodiments, it is desirable that the non-porous, polymeric film may have a yield strength greater than about 10 MPa depending on the type and thickness of material. A material having a lower yield strength may be too stretchable and, therefore, more susceptible to breaking with the application of small amounts of compression and/or apposition forces.

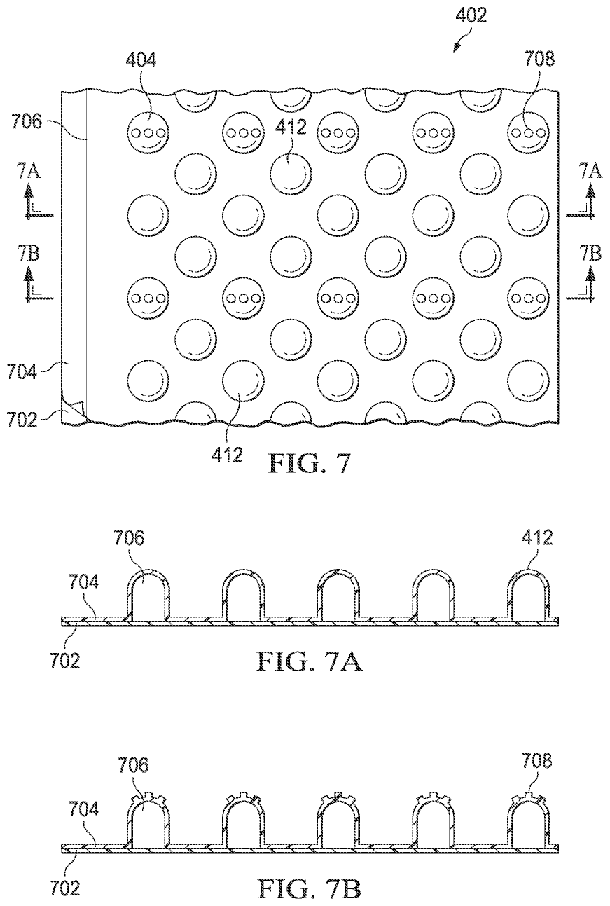

FIG. 7 is a plan view of the first layer 402 of the first bridge 212, illustrating additional details that may be associated with some embodiments. FIG. 7A is a section view of the first layer of the first bridge taken along line 7A-7A of FIG. 7, illustrating additional details that may be associated with some embodiments of the first bridge 212. In some embodiments, the first layer 402 of the first bridge 212 may include the closed cells 412 formed from a web of polymeric film. In some embodiments, the first layer 402 may comprise two sheets of polymeric film, such as a first sheet 702 and a second sheet 704. The first sheet 702 and the second sheet 704 may have inner surfaces coupled to each other in a pattern defining the plurality of the closed cells 412. The first sheet 702 and the second sheet 704 may be sealed to each other to form a sealed region 706 defining the closed cells 412. The closed cells 412 and the sealed region 706 may be formed from a process such as, for example, vacuum forming. In some embodiments, the sealed region 706 may be formed by a heat seal between the inner surfaces of the first sheet 702 and the second sheet 704, while the closed cells 412 may be formed simultaneously by vacuum forming. In another example embodiment, the sealed region 706 may be formed by adhesion between the first sheet 702 and the second sheet 704. Alternatively, the first sheet 702 and the second sheet 704 may be adhesively bonded to each other. The sealed region 706 may be flexible enough so that the first bridge 212 is sufficiently flexible to conform to the shape of the tissue site 202. The sealed region 706 may be sufficiently flexible or sized so that the first bridge 212 may be folded to conform to the tissue site 202 to provide optimal negative pressure to the tissue site 202.

In some embodiments, the closed cells 412 may be substantially airtight to inhibit collapsing of the closed cells 412 from the application of negative pressure which could block the flow of fluid through the dressing interface 120. The closed cells 412 may be substantially airtight when formed and have an internal pressure that is an ambient pressure. In another example embodiment, the closed cells 412 may be inflated with air or other suitable gases such as, for example, carbon dioxide or nitrogen. The closed cells 412 may be inflated to have an internal pressure greater than the atmospheric pressure to maintain their shape and resistance to collapsing under pressure and external forces. For example, the closed cells 412 may be inflated to a pressure up to about 25 psi above the atmospheric pressure so that they do not collapse as described above.

The polyurethane film may have a thickness within a range of 400 to 1100 microns. In some example embodiments, the first layer 402 and the second layer 404 of the first bridge 212, including the closed cells 412, may be formed from thermoplastic polyurethane film having a thickness of 500 microns. In some example embodiments, the first sheet 702 and the second sheet 704, prior to fabricating the first layer 402 and the second layer 404 of the first bridge 212, may each have a thickness of about 500 μm to about 1000 μm. In some embodiments, the first sheet 702 and the second sheet 704 may each have a thickness of about 500 μm. In some embodiments, the thickness of the layer that does not have the closed cells may be up to 50% thinner than the thickness of the layer that that includes the closed cells. For example, the thickness of the second layer 404 without any closed cells may be up to 50% thinner than the thickness of the first layer 402 that has the closed cells 412. After the layers have been fabricated, the sealed region 706 may have a thickness between about 800 μm and about 3000 μm. If the fabrication process comprises injection molding, the closed cells 412 may have a thickness between about 250 μm and about 1000 μm. If the closed cells 412 are fabricated by drawing the polyurethane film to form the closed cells 412, the top portion of the closed cells 412 may have a thickness as thin as 50 μm.

After the closed cells 412 have been fabricated, the walls of the closed cells 412 may have a thickness relative to the thickness of the first sheet 702 and the second sheet 704 as defined by a draw ratio, i.e., the ratio of the average height of the closed cells 412 to the average thickness of the first sheet 702 and the second sheet 704. In some example embodiments, the closed cells 412 may have a generally tubular shape as described above that may have been formed from the first sheet 702 and the second sheet 704 having various thicknesses and draw ratios. In some example embodiments, the first sheet 702 and the second sheet 704 may have an average thickness of 500 μm and the closed cells 412 may have an average height in a range between about 2.0 mm and 5.0 mm. Consequently, the closed cells 412 may have a draw ratio ranging from about 4:1 to about 10:1. In another example embodiment, the draw ratio may range from about 5:1 to about 13:1 where the thickness of the first sheet 702 and the second sheet 704 is an average of about 400 μm. In yet another example embodiment, the draw ratio may range from about 3:1 to about 9:1 where the thickness of the first sheet 702 and the second sheet 704 is an average of about 600 μm. In some embodiments, the closed cells 412 may have an average height in a range between about 1.0 mm and 4.0 mm depending on the thickness of the first sheet 702 and the second sheet 704. In some other embodiments, the closed cells 412 may have an average height in a range between about 2.0 mm and 5.0 mm depending on the thickness of the first sheet 702 and the second sheet 704. The first sheet 702 and the second sheet 704 may each have the same or different thicknesses and flexibilities, but are substantially non-stretchable as described above so that the closed cells 412 maintain a generally constant volume without bursting after a compression force is applied to the dressing interface 120 or negative pressure is applied to the dressing interface 120 and the tissue site 202. Consequently, even when a load is applied to the dressing interface 120 which squeezes the closed cells 412 into a different shape, the closed cells 412 are sufficiently flexible to recover their original shape after being squeezed without bursting.

In some embodiments, the closed cells 412 generally have a volumetric shape that is hemispherical or tubular. The closed cells 412 may have a circular base having an average diameter between about 1.0 mm and about 10 mm. In some other embodiments, the closed cells 412 may have a diameter between about 2.0 mm and about 5.0 mm. In some embodiments, the closed cells 412 also may have a pitch, i.e., the center to center distance between each of the closed cells 412, between about 1 mm and 10 mm. In some other embodiments, the closed cells 412 may also have a pitch between about 2 mm and about 3 mm. Because the sealed region 706 defines the base of the closed cells 412 including the diameter of a circular base and the pitch of closed cells 412, the surface area of the first layer 402 covered by the closed cells 412 may also be determined as a percentage, i.e., the cell coverage percentage. In one example embodiment wherein the diameter of the closed cells 412 is about 1.0 mm and the pitch is about 2.0 mm, the cell coverage percentage is about 22% of the surface area of the first layer 402. In another example embodiment wherein the diameter of the closed cells 412 is about 2.0 mm and the pitch is about 5.0 mm, the cell coverage percentage is about 14% of the surface area of the first layer 402. In yet another example embodiment wherein the diameter of the closed cells 412 is about 1.5 mm, the pitch is about 2.0 mm, and the closed cells 412 are more tightly arranged such that there are about 28.5 cells in a 10 mm² section of the first layer 402, the cell coverage percentage is about 51% of the surface area of the first layer 402. Depending on the diameter, pitch, and arrangement of the closed cells 412, the cell coverage percentage may range between about 10% and about 60% of the surface area of either one of the layers having the closed cells such as the first layer 402. Closed cells 412 having other base shapes or volumetric shapes also may have a cell coverage percentage in generally the same range.

FIG. 7B is a section view of the first layer of the first bridge taken along line 7B-7B of FIG. 7, illustrating additional details that may be associated with some embodiments. In some embodiments, a portion of those closed cells 412 extending through the aperture 408 of the first bridge 212 may be textured with surface features, which may be protrusions or indentations, to enhance fluid flow through the first bridge 212 to the tissue interface 108 and the tissue site 202 as described above. In some embodiments, as shown in FIG. 7 and FIG. 7B, closed cells 412 may be embossed with projections or nodes, such as nodes 708, so that the nodes 708 on top of the closed cells 412 contact the tissue interface 108 to enhance fluid flow to tissue site 202. The projections or nodes 708 may have a flexibility similar to the closed cells 412.

Additionally, the construction of the first layer 402 and the closed cells 412 of the first bridge 212 described above in connection with FIGS. 7-7B may also apply to the construction of the first layer 502 and the closed cells 512 of the second bridge 214.

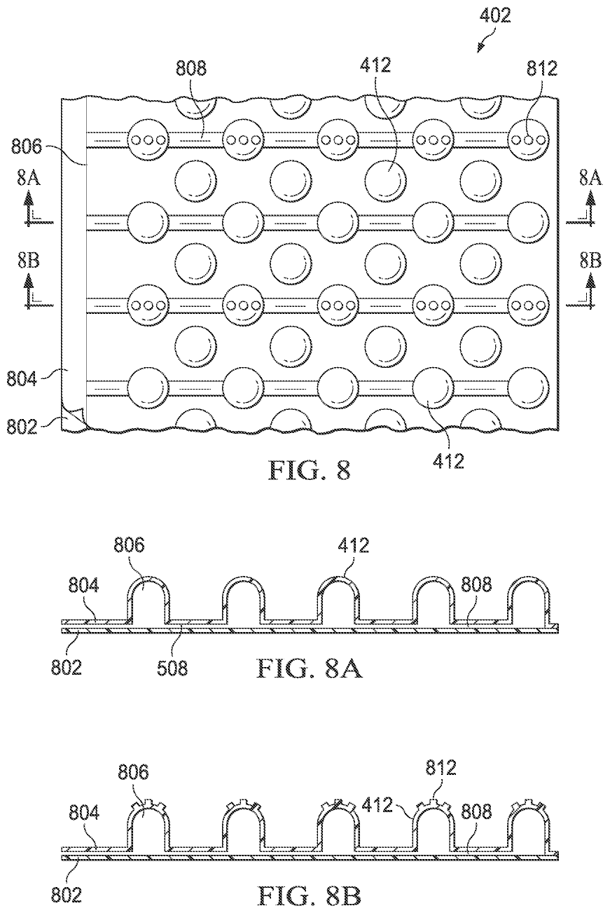

FIG. 8 is a plan view of another example of the first layer 402 of the first bridge 212 that may be associated with some embodiments of the dressing interface 120 of FIG. 3. FIG. 8A is a section view of the first layer of the first bridge taken along line 8A-8A of FIG. 8, illustrating additional details that may be associated with some embodiments. In some embodiments, the first layer 402 may comprise chambers formed between the closed cells 412. The chambers may better distribute the apposition force resulting from the application of negative pressure applied the tissue interface 108 because the volume of the chambers is greater than the volume of the individual closed cells. In one exemplary embodiment shown in FIGS. 8 and 8A, the first layer 402 may comprise a first sheet 802 and a second sheet 804 of polymeric film having inner surfaces coupled to each other in a pattern defining the plurality of the closed cells 412. The first sheet 802 and the second sheet 804 may be sealed to each other to form a sealed region 806 defining the closed cells 412. The first layer 402 may also comprise a plurality of passageways 808 fluidly coupling at least two of the closed cells 412 to form a closed chamber. In one exemplary embodiment, a closed chamber is formed by the closed cells 412 in a row fluidly coupled by the passageways 808, as shown in FIG. 8. The closed chambers may be formed in alternating rows as also shown in FIG. 8. The formation of closed chambers with closed cells in any pattern distributes apposition forces applied to the first layer 402 more equally across the first layer 402.

FIG. 8B is a section view of the first layer of the first bridge taken along line 8B-8B of FIG. 8, illustrating additional details that may be associated with some embodiments. In some embodiments, a portion of those closed cells 412 extending through the aperture 508 of the first bridge 212 may be textured with surface features, which may be protrusions or indentations, to enhance fluid flow through the first bridge 212 to the tissue interface 108 and the tissue site 202 as described above. In some embodiments, as shown in FIG. 8 and FIG. 8B, closed cells 412 may be embossed with projections or nodes, such as nodes 812, so that the nodes 812 on top of the closed cells 412 contact the tissue interface 108 to enhance fluid flow to tissue site 202.

Additionally, the construction of the first layer 402 and the closed cells 412 of the first bridge 212 described above in connection with FIGS. 8-8B may also apply to the construction of the first layer 502 and the closed cells 512 of the second bridge 214.

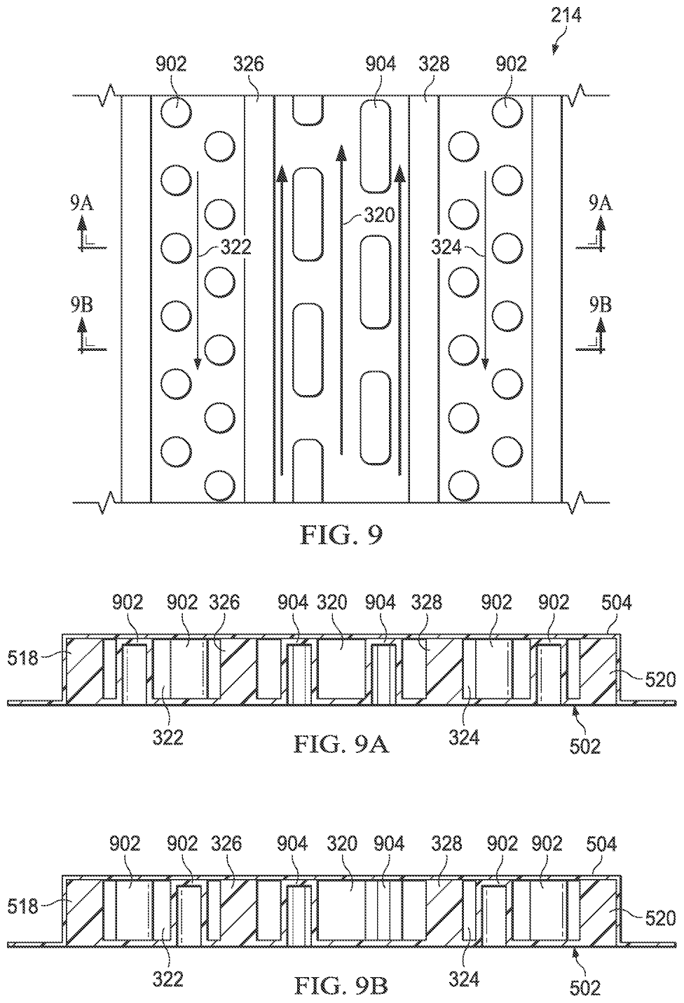

FIG. 9 is a plan view of the second bridge 214, illustrating additional details that may be associated with some embodiments. As indicated above, the second bridge may comprise a plurality of closed cells 512 sized and arranged in different patterns within the sealed space. The closed cells 512 of the second bridge 214 may have a variety of shapes and be sized arranged in different patterns within the second sealed space 506. For example, the second bridge 214 may comprise at least two sets of closed cells 512, such as a first plurality of closed cells 902 and a second plurality of closed cells 904. In some embodiments, the first plurality of closed cells 902 may have a generally cylindrical shape and the first plurality of closed cells 902 may have a generally rectangular shape. In some embodiments, each of the third fluid pathway 322 and the fourth fluid pathway 324 may include two rows of the first plurality of closed cells 902, and the second fluid pathway 320 may include two rows of the second plurality of closed cells 904. In some embodiments, the first plurality of closed cells 902 in a first row may be offset or staggered from the first plurality of closed cells 902 in a second row, and the second plurality of closed cells 904 in a first row may be offset or staggered from the second plurality of closed cells 904 in a second row.

FIG. 9A is a section view of the second bridge 214 of FIG. 9 taken along line 9A-9A, and FIG. 9B is a section view of the second bridge 214 of FIG. 9 taken along line 9B-9B, illustrating additional details that may be associated with some embodiments. In some embodiments, the second plurality of closed cells 904 may form the second fluid pathway 320 between the first barrier 326 and the second barrier 328. In some embodiments, the third fluid pathway 322 may be formed between the first plurality of closed cells 902, the first barrier 326, and the first seal 518. The fourth fluid pathway 324 may be formed between the first plurality of closed cells 902, the second barrier 328, and the second seal 520. In some embodiments, the first barrier 326, the second barrier 328, the first seal 518, and the second seal 520 may be formed by welding the first layer 502 to the second layer 504.

In some embodiments, the second plurality of closed cells 904 disposed in the second fluid pathway 320 may be larger and have a larger pitch than the first plurality of closed cells 902 disposed in the third fluid pathway 322 and the fourth fluid pathway 324 to increase fluid flow of instillation fluid being applied to the tissue interface 108. The first plurality of closed cells 902 disposed in the third fluid pathway 322 and the fourth fluid pathway 324 may have a noticeably smaller diameter and pitch than the larger second plurality of closed cells 904 that may restrict fluid flow to facilitate pressure sensing within the second sealed space 506. The arrangement and dimensions of the first plurality of closed cells 902 and the second plurality of closed cells 904 may be tailored to manage the delivery of instillation fluid to the tissue interface 108 and the measurement of pressure within the second sealed space 506.

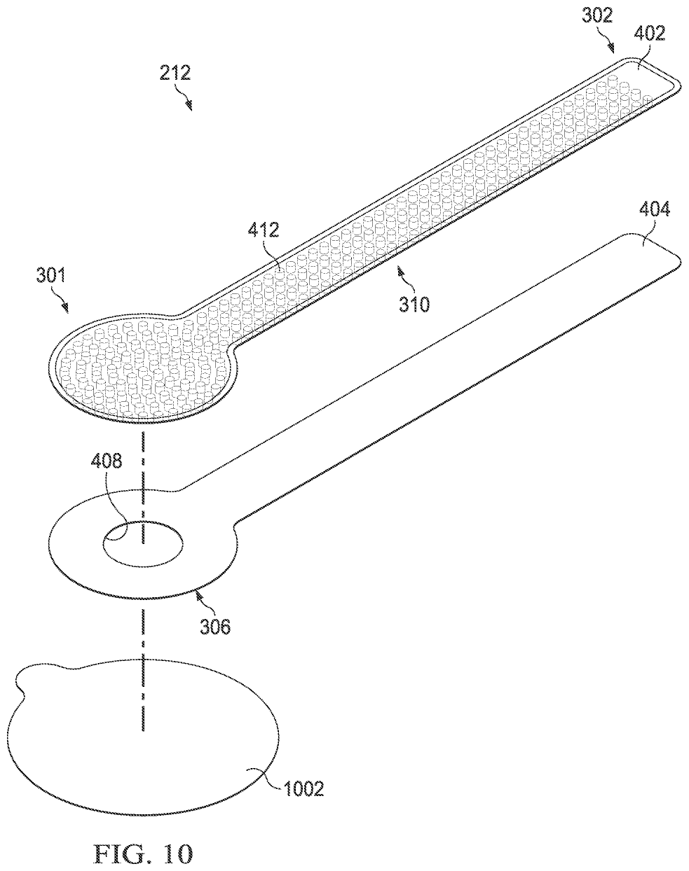

FIG. 10 is a perspective assembly view of a portion of the first bridge 212, illustrating additional details that may be associated with some embodiments. In some embodiments, the outer surface of the second layer 404 may be covered with a removable cover layer 1002 at the first end 301 of the first bridge 212. For example, the removable cover layer 1002 may cover the aperture 408 in the first applicator 306 of the first bridge 212. The removable cover layer 1002 may be removed prior to fluidly coupling the first bridge 212 to the tissue interface 108. In other embodiments, removal of the removable cover layer 1002 may reveal an adhesive on the outer surface of the second layer 404. The adhesive may be used to adhere the first bridge 212 to the cover 110 of the dressing 104.

In other embodiments, the first bridge 212 may include a contact layer. The contact layer may be coupled to the outer surface of the second layer 404 and be configured to contact the epidermis 204. The contact layer may wick fluid away from the epidermis 204 to prevent maceration. For example, the contact layer may comprise a wicking layer to prevent skin maceration.

In other embodiments, the first bridge 212 may further include an intermediate layer positioned between the first layer 402 and the second layer 404. The intermediate layer may have a first surface and a second surface opposite the first surface, with a plurality of features extending from one or both surfaces of the intermediate layer. The plurality of features extending from the first surface of the intermediate layer may be opposingly aligned with the plurality of features extending from the second surface of the intermediate layer. The first surface of the intermediate layer may be coupled to the inner surface of the first layer 402 and the second surface of the intermediate layer may be coupled to the inner surface of the second layer 404. In other embodiments, the plurality of features extending from the first surface may form a corresponding void in the second surface of the intermediate layer and the plurality of features extending form the second surface may form a corresponding void in the first surface of the intermediate layer. In some embodiments, the plurality of features extending from one or both surfaces of the intermediate layer may comprise the first plurality of features 310 of the first bridge 212.

In some embodiments, the first layer 402 and the second layer 404 of the first bridge 212 may be transparent or light-occlusive. If not transparent, they may have a range of colors, including white. Different layers may be different colors or, alternatively, transparent to improve visibility of contents in the dressing interface 120.

In other embodiments, the first bridge 212 may comprise multiple pneumatic interface connections, or apertures, to allow the first bridge 212 to be placed in various locations. Each of the apertures may be configured to be coupled to the cover 110 and fluidly coupled to the tissue interface 108. In some embodiments, each of the apertures may comprise a removable cover layer, like the removable cover layer 1002, to seal each of the apertures when not in use. The apertures may allow for more efficient treatment of wounds. For example, a clinician may position the apertures in various locations on the wound, or may position the apertures across multiple wounds.

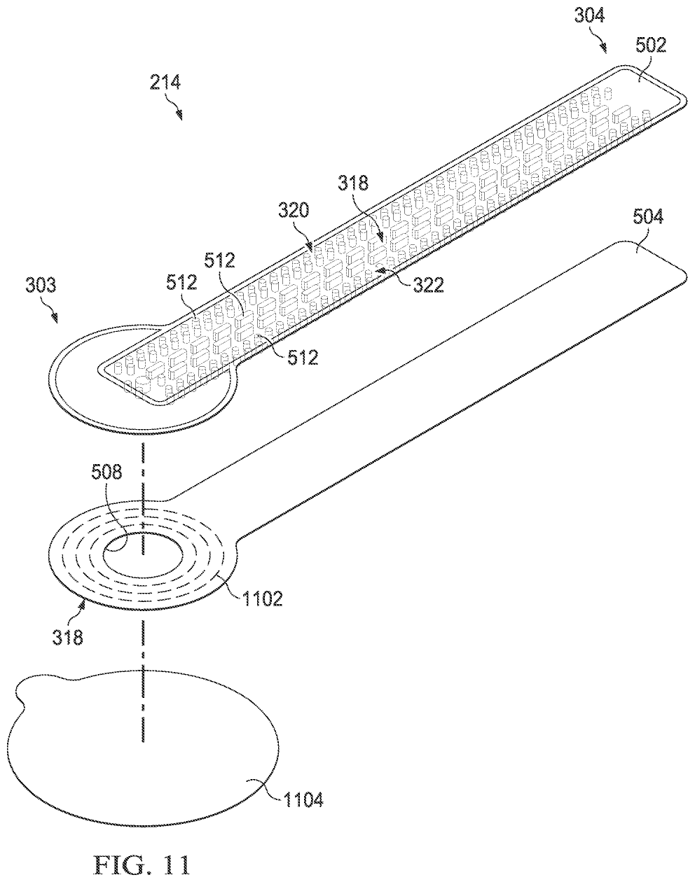

FIG. 11 is a perspective assembly view of a portion of the second bridge 214, illustrating additional details that may be associated with some embodiments. The outer surface of the second layer 404 may be covered with a removable cover layer 1104 at the first end 303 of the second bridge 214. For example, the removable cover layer 1104 may cover the aperture 508 in the second applicator 318 of the second bridge 214. The removable cover layer 1104 may be removed prior to fluidly coupling the second bridge 214 to the tissue interface 108. In other embodiments, removal of the removable cover layer 1104 may reveal an adhesive on the outer surface of the second layer 504. The adhesive may be used to adhere the second bridge 214 to the cover 110 of the dressing 104. The adhesive may be used to adhere the second bridge 214 to the tissue interface 108.

In some embodiments, the second layer 504 may include a plurality of fenestrations 1102 at the first end 303 that surround the aperture 508. For example, the plurality of fenestrations 1102 may be disposed in the second layer 504 of the second applicator 318 surrounding the aperture 508. In some embodiments, the aperture 508 may be in fluid communication with the second fluid pathway 320 and the plurality of fenestrations 1102 may be in fluid communication with both the third fluid pathway 322 and the fourth fluid pathway 324. In some embodiments, the plurality of fenestrations 1102 may act as a micro valve to restrict the interference of fluid and prevent blockages. The aperture 508 and the fenestrations 1102 may also be covered by the removable cover layer 1104.

In other embodiments, the second bridge 214 may include a contact layer. The contact layer may be coupled to the outer surface of the second layer 504 and be configured to contact the epidermis 204. The contact layer may wick fluid away from the epidermis 204 to prevent maceration. For example, the contact layer may comprise a wicking layer to prevent skin maceration.

In still other embodiments, the second bridge 214 may further include an intermediate layer positioned between the first layer 502 and the second layer 504. The intermediate layer may have a first surface and a second surface, with a plurality of features extending from one or both of the first surface and the second surface. The plurality of features extending from the first surface may be opposingly aligned with the plurality of features extending from the second surface. The first surface of the intermediate layer may be coupled to the inner surface of the first layer 502 and the second surface of the intermediate layer may be coupled to the inner surface of the second layer 504. In other embodiments, the plurality of features extending from the first surface may form a corresponding void in the second surface of the intermediate layer and the plurality of features extending form the second surface may form a corresponding void in the first surface of the intermediate layer. In some embodiments, the plurality of features extending from one or both surfaces of the intermediate layer may comprise the second plurality of features 330, the third plurality of features 332, and the fourth plurality of features 334 of the second bridge 214.

In some embodiments, the first layer 502 and the second layer 504 of the second bridge 214 may be transparent or light-occlusive. If not transparent, they may have a range of colors, including white. Different layers may be different colors or, alternatively, transparent to improve visibility of contents in the dressing interface 120.

In other embodiments, the second bridge 214 may comprise multiple pneumatic interface connections, or apertures, to allow the first bridge to be placed in various locations. Each of the apertures may be configured to be coupled to the cover 110 and to be fluidly coupled to the tissue interface 108. In some embodiments, each of the apertures may comprise a cover layer, like the removable cover layer 1104, to seal each of the apertures when not in use. The apertures may allow for more efficient treatment of wounds. For example, the clinician may position the apertures in various locations on the wound, or may position the apertures across multiple wounds.

In operation, negative pressure may be supplied to the tissue interface 108 by the first bridge 212. Negative pressure supplied from the negative-pressure source 102 of the therapy unit 200 may travel through the negative-pressure delivery conduit 130 to the first bridge 212 for delivery to the tissue interface 108, as shown in FIG. 2. In some embodiments, the negative-pressure source 102 of the therapy unit 200 may be fluidly coupled to the first bridge via the multi-lumen conduit 338, as shown in FIG. 3. The multi-lumen conduit 338 may comprise the negative-pressure delivery conduit 130 and the pressure sensing conduit 134. In such embodiments, negative pressure supplied from the negative-pressure source 102 of the therapy unit 200 may travel through the multi-lumen conduit 338 to the connector 336. The connector 336 may separate the negative-pressure delivery conduit 130 from the pressure sensing conduit 134 and fluidly couple the negative-pressure delivery conduit 130 to the first bridge conduit 304. Negative pressure is then supplied to the tissue interface 108 through the first bridge conduit 304, the first fluid pathway 308 of the first bridge 212, and the aperture 408 of the first applicator 306.

Instillation fluid may be delivered to the tissue interface 108 by the second bridge 214. Instillation fluid supplied from the solution source 118 of the therapy unit 200 may travel through the instillation delivery conduit 140 to the second bridge 214 for delivery to the tissue interface 108, as shown in FIG. 2. In some embodiments, the instillation delivery conduit 140 may pass through the connector 336 for fluid coupling to the second bridge 214, as shown in FIG. 3. The instillation fluid may then travel through the interface 342, the second fluid pathway 320, and the aperture 508 of the second applicator 318 for delivery to the tissue interface 108. The instillation fluid may then flow across the tissue site 202 from the second bridge 214 positioned at a first location on the tissue interface 108 to the first bridge 212 positioned at a second location on the tissue interface 108. The instillation fluid and wound exudate may be removed by the negative-pressure source 102 through the first bridge 212. In some embodiments, the wound exudate and instillation fluid removed from the tissue site 202 may be collected in the container 106 of the therapy unit 200.

Pressure may be measured at the tissue interface 108 by the second bridge 214. In some embodiments, the therapy unit 200 may include sensors for monitoring pressure at the tissue site 202. In some embodiments, the sensors may be fluidly coupled to the tissue interface 108 via the multi-lumen conduit 338, as shown in FIG. 3. The multi-lumen conduit 338 may comprise the negative-pressure delivery conduit 130 and the pressure sensing conduit 134. In such embodiments, the connector 336 may separate the pressure sensing conduit 134 from the negative-pressure delivery conduit 130 and fluidly couple the pressure sensing conduit 134 to the second bridge conduit 314. The second bridge conduit 314 may be fluidly coupled to the third fluid pathway 322 and the fourth fluid pathway 324. The third fluid pathway 322 and the fourth fluid pathway 324 may also be fluidly coupled to the second applicator 318 for sensing pressure at the tissue interface 108.

In some embodiments, pressure sensing can be conducted through the third fluid pathway 322 and the fourth fluid pathway 324 of the second bridge 214. Instillation therapy can be provided through the second fluid pathway 320, and negative-pressure therapy can be provided through the first fluid pathway 308. As illustrated in FIG. 2, the first bridge 212 can be positioned at the first end 220 of the cover 110 and the second bridge 214 can be positioned at the second end 224 of the cover 110. Preferably, the first end 220 and the second end 224 may be spaced apart from each other. Negative-pressure therapy can be provided through the first fluid pathway 308 first bridge 212 at the first end 220, and pressure sensing can be conducted through the third fluid pathway 322 and the fourth fluid pathway 324 at the second end 224. Negative-pressure therapy and pressure sensing can be conducted at opposite ends of the tissue site 202. Separating pressure sensing and negative-pressure therapy permits the therapy unit 200 to provide therapeutic pressure across the tissue site and limits instances of localized pressure readings that may not accurately represent the pressure provided at the tissue site 202. Furthermore, separating pressure sensing and negative-pressure therapy permits the therapy unit 200 to determine that both the first bridge 212 and the second bridge 214 are pneumatically coupled to the tissue interface 108.

In some embodiments, fluids instilled during an instillation therapy cycle may create a large bolus of fluid at the tissue site. As the fluid is drawn off at the initiation of negative-pressure therapy through the first bridge 212, the instillation fluid will not be drawn across the third fluid pathway 322 and the fourth fluid pathway 324. Consequently, the pressure sensing conducted through the second bridge 214 can determine pressure at the tissue site 202 free from interference of the instillation fluid bolus. The connector 336 can permit separation of the pressure sensing functions from negative-pressure therapy, allowing using of many existing therapy units that provide pressure sensing and negative-pressure therapy through a single multi-lumen tube. The connector 336 can fluidly couple pressure sensing lumens of a multi-lumen conduit to the second bridge 214 while fluidly coupling the negative-pressure delivery lumens of the multi-lumen conduit to the first bridge 212. In this manner, the first bridge 212 and the second bridge 214 may be used many therapy units.

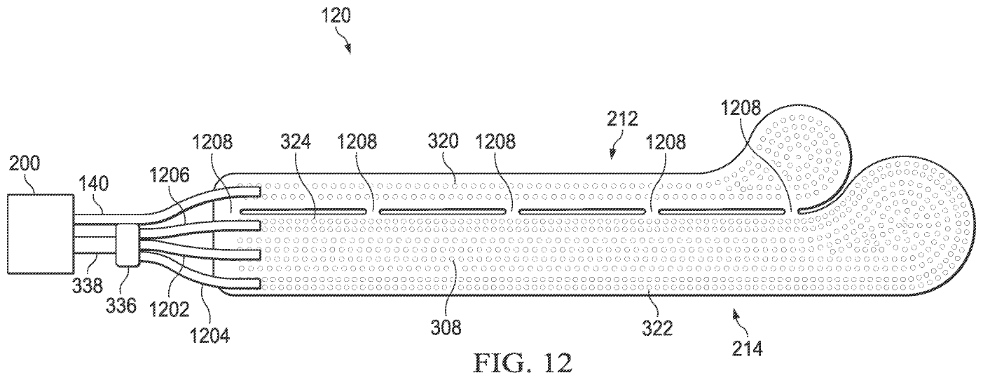

FIG. 12 is a schematic diagram of the dressing interface 120, illustrating additional details that may be associated with some embodiments. In some embodiments, the first bridge 212 may comprise an instillation pathway, such as the second fluid pathway 320, and the second bridge 214 may comprise a negative pressure pathway, such as the first fluid pathway 308, and at least one sensing pathway, such as the third fluid pathway 322 and the fourth fluid pathway 324. The third fluid pathway 322 and the fourth fluid pathway 324 may be positioned outbound of the first fluid pathway 308 of the second bridge 214. In some embodiments, the first fluid pathway 308 may be fluidly coupled to the therapy unit 200 via a negative pressure conduit 1202, the second fluid pathway 320 may be fluidly coupled to the therapy unit 200 via the instillation delivery conduit 140, the third fluid pathway 322 may be fluidly coupled to the therapy unit 200 via a first sensing conduit 1204, and the fourth fluid pathway 324 may be fluidly coupled to the therapy unit 200 via a second sensing conduit 1206. In some embodiments, the instillation delivery conduit 140, the negative pressure conduit 1202, the first sensing conduit 1204, and the second sensing conduit 1206 may each be directly welded within the first bridge 212 or the second bridge 214. In other embodiments, the instillation delivery conduit 140, the negative pressure conduit 1202, the first sensing conduit 1204, and the second sensing conduit 1206 may be fluidly coupled to the first bridge 212 or the second bridge 214 via an interface pad, such as the interface 342 of FIG. 3 or the interface 407 of FIG. 4.

In some embodiments, the negative pressure conduit 1202, the first sensing conduit 1204, and the second sensing conduit 1206 may be fluidly coupled to the connector 336. The connector 336 may fluidly couple the negative pressure conduit 1202, the first sensing conduit 1204, and the second sensing conduit 1206 to the therapy unit 200 via the multi-lumen conduit 338. In some embodiments, the instillation delivery conduit 140 may bypass the connector 336 and be independently fluidly coupled to the therapy unit 200. In such embodiments, the instillation delivery conduit 140 may directly fluidly couple the solution source 118 of the therapy unit 200 to the second fluid pathway 320. In other embodiments, the instillation delivery conduit 140 may be fluidly coupled to the connector 336. In such embodiments, the connector 336 may fluidly isolate the instillation delivery conduit 140 from the negative pressure conduit 1202, the first sensing conduit 1204, and the second sensing conduit 121. The connector 336 may also fluidly couple the instillation delivery conduit 140 to the therapy unit 200. In other embodiments, the connector 336 may fluidly couple the instillation delivery conduit 140 to the therapy unit 200 via another single lumen conduit fluidly coupled to the connector 336.

In some embodiments, the first bridge 212 may be releasably coupled to the second bridge 214. For example, a series of perforations, slots, tabs, or tearable sections may be disposed along a length of the first bridge 212 and the second bridge 214 to releasably couple the first bridge 212 to the second bridge 214. In some embodiments, the series of perforations, slots, tabs, or tearable sections may be positioned within welds fluidly isolating the first bridge 212 and the second bridge 214. In some embodiments, the first bridge 212 may comprise the second fluid pathway 320 and the second bridge 214 may comprise the first fluid pathway 308, the third fluid pathway 322, and the fourth fluid pathway 324. The plurality of tabs 1208 may be positioned between the second fluid pathway 320 and the fourth fluid pathway 324 to releasably couple the first bridge 212 to the second bridge 214.

In some embodiments, the plurality of tabs 1208 may be comprised of the series of perforations having between about 7 and about 10 tabs or ties per inch (TPI). In some embodiments a length of each of the plurality of tabs 1208 may be between about 0.5 mm and about 2 mm. In some embodiments, each of the plurality of tabs 1208 may be separated by a distance of about 0.8 mm to about 12 mm. In some embodiments, the plurality of tabs 1208 may have a thickness between about 0.25 mm to about 1 mm. In some embodiments, the plurality of tabs 1208 may comprise the same material as the first layer 402, the first layer 502, the second layer 404, the second layer 504, the closed cells 412, and the closed cells 512, discussed above. For example, the plurality of tabs 1208 may comprise a polymeric film such as a thermoplastic polyurethane (TPU) film that is permeable to water vapor but impermeable to liquid. Alternatively, the plurality of tabs 1208 may be cut into a non-woven wicking substrate applied to a tissue-facing side of the first bridge 212 and the second bridge 214. In some embodiments, the non-woven wicking substrate may be a coating that prevents skin maceration underneath the first bridge 212 and the second bridge 214.

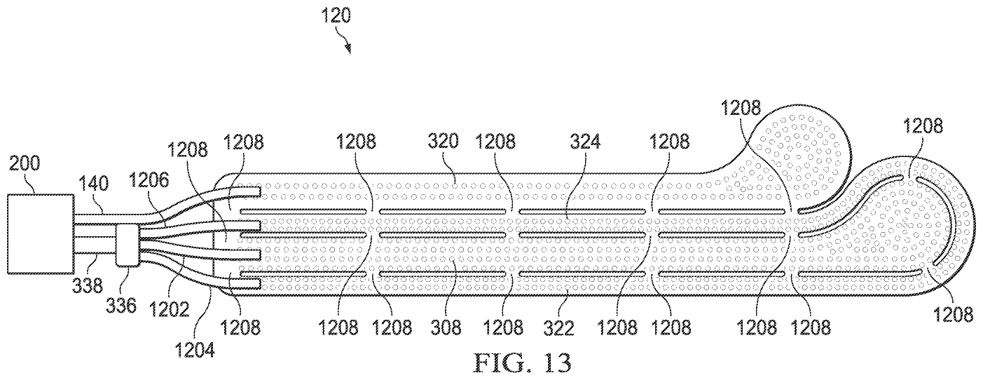

FIG. 13 is a schematic diagram of the dressing interface 120 of FIG. 12, illustrating additional details that may be associated with some embodiments. In some embodiments, the series of perforations, slots, tabs, or tearable sections, such as the plurality of tabs 1208, may be positioned between each of the fluid pathways within the first bridge 212 and the second bridge 214 to fluidly isolate the fluid pathways. For example, the plurality of tabs 1208 may be between the second fluid pathway 320 and the fourth fluid pathway 324, between the fourth fluid pathway 324 and the first fluid pathway 308, and between the first fluid pathway 308 and the third fluid pathway 322. Such embodiments allow a user to target specific desired locations on a wound for treatment. For example, the first fluid pathway 308, the second fluid pathway 320, the third fluid pathway 322, and the fourth fluid pathway 324 may be fluidly coupled to a wound at various locations to provide maximum efficiency for negative-pressure, instillation therapy, and pressure determination at the tissue site. In some example embodiments, the first fluid pathway 308 may be fluidly coupled to the bottom of the wound and the second fluid pathway 320, the third fluid pathway 322, and the fourth fluid pathway 324 may be fluidly coupled to the top of the wound to ensure therapeutic pressures across the wound and to allow gravity to aid in delivering instillation fluid. Alternatively, the first fluid pathway 308, the third fluid pathway 322, and the fourth fluid pathway 324 may be fluidly coupled to the top of the wound and the second fluid pathway 320 may be fluidly coupled to the bottom of the wound to maximize the amount of time the instillation fluid contacts the wound as it works against gravity. In still other example embodiments, the first bridge 212 and the second bridge 214 may be fluidly coupled to the wound without separating any of the second fluid pathway 320, the first fluid pathway 308, the third fluid pathway 322, or the fourth fluid pathway 324.

In some embodiments, the second fluid pathway 320, the fourth fluid pathway 324, the first fluid pathway 308, and the third fluid pathway 322 may be fluidly coupled to the therapy unit 200 via the instillation delivery conduit 140, a negative pressure conduit 1202, a first sensing conduit 1204, and a second sensing conduit 1206, respectively. In some embodiments, the instillation delivery conduit 140, the negative pressure conduit 1202, the first sensing conduit 1204, and the second sensing conduit 1206 may each be directly welded within the first bridge 212 or the second bridge 214. In other embodiments, the instillation delivery conduit 140, the negative pressure conduit 1202, the first sensing conduit 1204, and the second sensing conduit 1206 may be fluidly coupled to the first bridge 212 or the second bridge 214 via an interface pad, such as the interface 342 of FIG. 3.

In some embodiments, the negative pressure conduit 1202, the first sensing conduit 1204, and the second sensing conduit 1206 may be fluidly coupled to the connector 336. The connector 336 may fluidly couple the negative pressure conduit 1202, the first sensing conduit 1204, and the second sensing conduit 1206 to the therapy unit 200 via the multi-lumen conduit 338. In some embodiments, the instillation delivery conduit 140 may bypass the connector 336 and be independently fluidly coupled to the therapy unit 200. In such embodiments, the instillation delivery conduit 140 may directly fluidly coupled the solution source 118 of the therapy unit 200 to the second fluid pathway 320. In other embodiments, the instillation delivery conduit 140 may be fluidly coupled to the connector 336. In such embodiments, the connector 336 may fluidly isolate the instillation delivery conduit 140 from the negative pressure conduit 1202, the first sensing conduit 1204, and the second sensing conduit 121. The connector 336 may also fluidly couple the instillation delivery conduit 140 to the therapy unit 200. In some embodiments, the connector 336 may fluidly couple the instillation delivery conduit 140 to the therapy unit 200 via another single lumen conduit fluidly coupled to the connector 336.

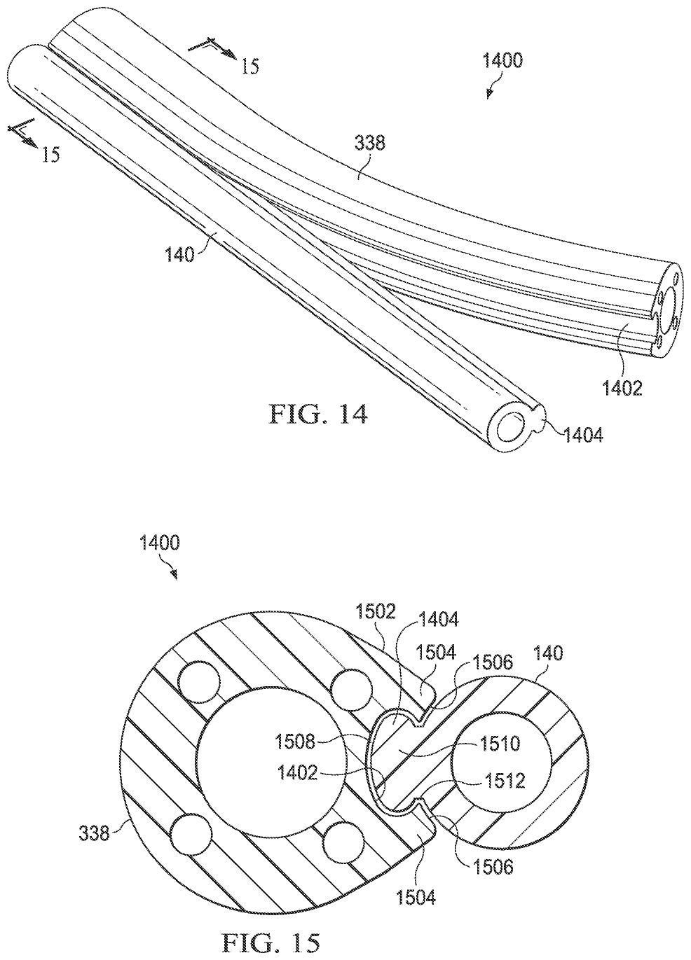

FIG. 14 is a perspective view of a conduit system 1400 that may be associated with some embodiments of the dressing interface 120 of FIG. 3. The conduit system 1400 may comprise at least two conduits, such as the instillation delivery conduit 140 and the multi-lumen conduit 338. In some embodiments, the instillation delivery conduit 140 may be a single lumen conduit. In other embodiments, the instillation delivery conduit 140 may be a multi-lumen conduit. In still other embodiments, the conduit system 1400 may comprise at least two single-lumen conduits or at least two multi-lumen conduits. In some embodiments, the instillation delivery conduit 140 may fluidly couple the therapy unit 200 to the first bridge 212 and the multi-lumen conduit 338 may fluidly couple the therapy unit 200 to the second bridge 214. For example, the instillation delivery conduit 140 may deliver instillation fluid to the first bridge 212 and the multi-lumen conduit 338 may provide negative pressure and pressure sensing to the second bridge 214. A length of the instillation delivery conduit 140 and the multi-lumen conduit 338 may be sufficiently long to allow for flexible placement of the dressing interface 120 at the tissue site. For example, the length of the instillation delivery conduit 140 and the multi-lumen conduit 338 may permit the dressing interface 120 to be positioned at a tissue site on a patient that is separated from the therapy unit 200. The patient may engage in some movement without disturbing the therapy unit 200.

In some embodiments, the instillation delivery conduit 140 may be configured to be coupled to the multi-lumen conduit 338 to allow a user or a patient to gather and manage the instillation delivery conduit 140 and the multi-lumen conduit 338. For example, the multi-lumen conduit 338 may comprise a groove 1402 and the instillation delivery conduit 140 may comprise a tongue 1404. In some embodiments, the tongue 1404 may run the length of the instillation delivery conduit 140 and the groove 1402 may run the length of the multi-lumen conduit 338. Generally, the groove 1402 may be disposed in a surface of the multi-lumen conduit 338 and be parallel to an axis of the multi-lumen conduit 338. Similarly, the tongue 1404 may project from a surface of the instillation delivery conduit 140 and be parallel to an axis of the instillation delivery conduit 140. The tongue 1404 of the instillation delivery conduit 140 may be configured to mate with the groove 1402 of the multi-lumen conduit 338. In some embodiments, at least a portion of the tongue 1404 may be inserted into at least a portion of the groove 1402 to couple at least a portion of the instillation delivery conduit 140 to the multi-lumen conduit 338. Mating of the tongue 1404 with the groove 1402 can permit a user to gather the instillation delivery conduit 140 and the multi-lumen conduit 338 by joining the conduits and preventing entanglement of the conduits with each other in a manner that may cause kinking or blocking of the conduits.

FIG. 15 is a cross-sectional view of the conduit system 1400 of FIG. 13 taken along line 15-15, illustrating additional details that may be associated with some embodiments. In some embodiments, the multi-lumen conduit 338 may include the groove 1402 formed in at least one side of the multi-lumen conduit 338. For example, the multi-lumen conduit 338 may have an enlarged portion 1502 running an axial length of the multi-lumen conduit 338. The groove 1402 may depend into the enlarged portion 1502 of the multi-lumen conduit 338. In some embodiments, the enlarged portion 1502 may include a pair of projecting walls 1504 defining the groove 1402. An outer edge 1506 of each of the projecting walls 1504 may depend toward each other to create a gap 1508 having a maximum width less than a maximum width of the groove 1402. In some embodiments, the tongue 1404 may project from a surface of the instillation delivery conduit 140. The tongue 1404 may have an ovoid shape. In some embodiments, the tongue 1404 may having a bulbous portion 1510 and a neck portion 1512. Each of the bulbous portion 1510 and the neck portion 1512 may extend an axial length of the instillation delivery conduit 140. In some embodiments, the bulbous portion may have a maximum width that is substantially equal to the maximum width of the groove 1402. Similarly, the neck portion 1512 may have a maximum width that is substantially equal to a maximum width of the gap 1508. The projecting walls 1504 and the tongue 1404 may be formed from a pliable material such as a silicone or polyurethane material. The projecting walls 1504 may flex, permitting the bulbous portion 1510 of the tongue 1404 to be pushed through the gap 1508 into the groove 1402. In some embodiments, the outer edge 1506 of the projecting walls 1504 may engage the neck portion 1512 to prevent inadvertent movement of the bulbous portion 1510 out of the groove 1402. In this manner, the groove 1402 may be configured to receive the tongue 1404 of the instillation delivery conduit 140. The tongue 1404 may be inserted into the groove 1402 to couple at least a portion of the instillation delivery conduit 140 to the conduit 215.

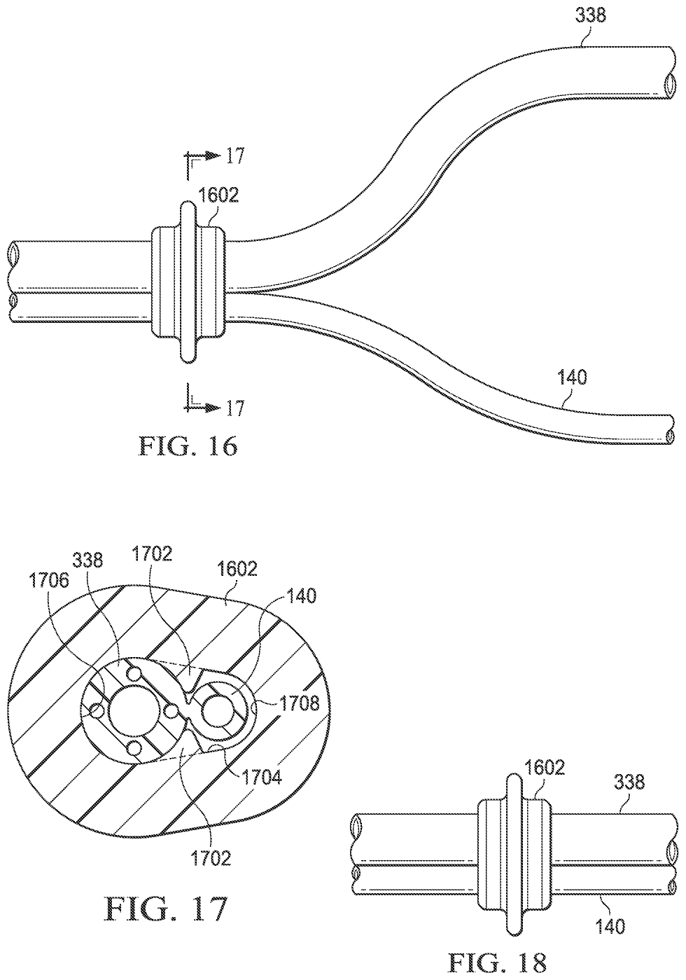

FIG. 16 is a schematic diagrams of a slip ring 1602 that may be associated with some embodiments of the dressing interface 120. In some embodiments, the dressing interface 120 may include a plurality of individual conduits or tubes fluidly coupling the dressing interface 120 to the therapy unit 200. The length of the plurality of individual conduits may be sufficiently long to allow for flexible placement of the dressing interface 120 at the tissue site. In some embodiments, the slip ring 1602 may be configured to gather and manage the plurality of individual conduits. For example, the slip ring 1602 may be configured to surround the multi-lumen conduit 338 and the instillation delivery conduit 140. The slip ring 1602 may couple at least a portion of the instillation delivery conduit 140 to the multi-lumen conduit 338. In some embodiments, the slip ring 1602 may allow the plurality of individual tubes to be moved without pulling on or dislodging the dressing interface 120. In other embodiments, the slip ring 1602 may be configured to hold the first bridge conduit 304, the second bridge conduit 314, and the instillation conduit 316. In still other embodiments, the slip ring 1602 may be configured to hold the instillation conduit 316, the negative pressure conduit 1202, the first sensing conduit 1204, and the second sensing conduit 1206.

In some embodiments, the dressing interface 120 may comprise a multi-lumen tube. The multi-lumen tube may be an extruded multi-lumen tube having at least two lumens side-by-side. In some embodiments, the at least two lumens of the extruded multi-lumen tube that are side-by-side may be configured to separate. For example, at least a portion of the at least two lumens of the multi-lumen tube may be separated into the instillation delivery conduit 140 and the multi-lumen conduit 338. In such embodiments, the slip ring 1602 may be configured to move the length of the multi-lumen conduit and couple at least a portion of the instillation delivery conduit 140 to the multi-lumen conduit 338 after the instillation delivery conduit 140 has been separated from the multi-lumen conduit 338.

FIG. 17 is a cross-sectional view of the slip ring 1602 taken along line 17-17 of FIG. 16, illustrating additional details that may be associated with some embodiments. In some embodiments, the slip ring 1602 may have an oval, circular, or amorphous shape. In some embodiments, the slip ring 1602 includes a hollow center 1704 configured to receive the instillation delivery conduit 140 and the multi-lumen conduit 338. In some embodiments, the hollow center 1704 may have an oval, circular, or amorphous shape. As illustrated in FIG. 17, the hollow center 1704 has a first end 1706 having a first effective diameter and a second end 1708 have a second effective diameter. In some embodiments, the first effective diameter may be larger than the second effective diameter. The first end 1706 and the second end 1708 may each have a semi-circular profile. The hollow center 1704 may further comprise a middle portion joining the first end 1706 and the second end 1708. The first end 1706 may be configured to receive a conduit having a larger diameter, such as the multi-lumen conduit 338. The second end 1708 may be configured to receive a conduit having a smaller diameter, such as the instillation delivery conduit 140. In other embodiments, the first end 1706 and the second end 1708 may have substantially equal effective diameters. The slip ring 1602 may further comprise at least two projections, such as projections 1702, extending from the middle portion of the hollow center 1704. The projections 1702 may be configured to engage the instillation delivery conduit 140 and the multi-lumen conduit 338 within the hollow center 1704. For example, the projections 1702 may be positioned closer to the second end 1708 than the first end 1706 to permit the first end 1706 to receive a larger diameter conduit than the second end 1708.

FIG. 18 is a schematic diagram of the slip ring 1602 of FIG. 16, illustrating additional details that may be associated with some embodiments. In some embodiments, the slip ring 1602 may be configured to couple an exterior surface of the instillation delivery conduit 140 to an exterior surface of the multi-lumen conduit 338. The slip ring 1602 may also be configured to slide along a length of the instillation delivery conduit 140 and the multi-lumen conduit 338. As the slip ring slides along the length of the instillation delivery conduit 140 and the length of the multi-lumen conduit 338, the slip ring may couple at least a portion of the instillation delivery conduit 140 to the multi-lumen conduit 338.

A method for manufacturing an apparatus for managing fluid from a tissue site is also disclosed. In one example embodiment, the method may comprise forming a first bridge and forming a second bridge. Forming a first bridge may comprise providing a first layer, providing a second layer, and coupling the first layer to the second layer. The first layer and the second layer may each have an outer surface and an inner surface. The first layer may have a first plurality of features extending from the inner surface. Coupling the first layer to the second layer may comprise coupling the inner surface of the second layer to the first layer, covering the plurality of features to form a first sealed space with the inner surface of the first layer. A first plurality of flow channels may be formed within the first sealed space. Such method may further comprise fluidly coupling a first bridge conduit to the first plurality of flow channels. Additionally or alternatively, the second layer may have an aperture configured to fluidly couple the first sealed space to the tissue site. In some embodiments, the first layer and the second layer may each comprise a polymeric film.

In some embodiments, forming a second bridge may comprise providing a third layer having a second plurality of features extending from a surface of the third layer, providing a fourth layer, and coupling the fourth layer to the third layer to cover the second plurality of surface features and form a second sealed space between the third layer and the fourth layer. The method may further comprise forming a first barrier and a second barrier between the third layer and the fourth layer. Additionally, the first barrier and the second barrier may define a second plurality of flow channels in the second sealed space between the first barrier and the second barrier, a third plurality of flow channels in the second sealed space between the first barrier and a first seal formed between a first portion of the third layer and a first portion of the fourth layer, and a fourth plurality of flow channels in the second sealed space between the second barrier and a second seal formed between a second portion of the third layer and a second portion of the fourth layer. The third plurality of flow channels and the fourth plurality of flow channels may be outbound of the second plurality of flow channels. In some embodiments, the method further comprises fluidly coupling a second bridge conduit to the second plurality of flow channels and fluidly coupling at least one sensing conduit to both the third plurality of flow channels and the fourth plurality of flow channels. In some embodiments, the third layer and the fourth layer may each comprise a polymeric film.

The method may further comprise coupling the first bridge conduit, the second bridge conduit, and the at least one sensing conduit to a connector block. Additionally or alternatively, the method may comprise providing a slip ring configured to hold the first bridge conduit, the second bridge conduit, and the at least one sensing conduit to each other. In other embodiments, the method may comprise forming a plurality of tabs along a length of the first bridge and releasably coupling the second bridge to the tabs.

In some additional embodiments, the method may comprise fluidly coupling the at least one sensing conduit and the first bridge conduit to a multi-lumen conduit having a central lumen and a plurality of peripheral lumens. In some embodiments, the at least one sensing conduit and the first bridge conduit may be fluidly coupled to the multi-lumen conduit via the connector block. The peripheral lumens may be fluidly coupled to the at least one sensing conduit, and the central lumen may be fluidly coupled to the first bridge conduit. Additionally or alternatively, the method may comprise forming a groove along a length of the multi-lumen conduit, forming a tongue along the length of the second bridge conduit, and mating the tongue with the groove.

The systems, apparatuses, and methods described herein may provide significant advantages. For example, the first bridge 212 and the second bridge 214 may maintain separate pathways for instillation and negative-pressure therapy, minimizing blockages and unintended siphoning of fluid during negative-pressure therapy treatment. Additionally, the first bridge 212 and the second bridge 214 may be placed at opposite sides of a wound to ensure the entire wound, rather than a single location on the wound, is subjected to negative pressure and instillation therapies. In other embodiments, the first bridge 212 and the second bridge 214 may be used to treat multiple wound sites at the same time.

Another advantage is that separating negative-pressure and sensing pathways allows the sensing pathways to be used to verify that the first bridge 212 and the second bridge 214 are in pneumatic connection with the dressing 104 and each other. Additionally, fluid flow will be away from the sensing pathway, preventing large volumes of fluid from contacting the port to the sensing pathway.

The dressing interface 120 may also be more comfortable for the patient. The low-profile design of the first bridge 212 and the second bridge 214 make the first bridge 212 and the second bridge 214 conformable and distributes external pressure to reduce pressure points and sores experienced by a patient. For example, the low-profile tubing connection reduces pressure points for a patient lying on the tubing or the dressing interface 120. Additionally, the low-profile design of the dressing interface 120 is highly resilient to blockages and thick exudate, and can also be used under compression.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 104, the container 106, or both may be eliminated or separated from other components for manufacture or sale. In other example configurations, the controller 112 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. An apparatus for managing fluid from a tissue site, the apparatus comprising:
a first portion having:
a first end configured to be fluidly coupled to a tissue interface;
a second end configured to be fluidly coupled to a first conduit; and
a first fluid pathway extending from the first end to the second end, the first fluid pathway comprising a first plurality of features projecting into the first fluid pathway;
a second portion having:
a first end configured to be fluidly coupled to the tissue interface;
a second end configured to be fluidly coupled to a second conduit;
a second fluid pathway formed along a length of the second portion, the second fluid pathway comprising a second plurality of features projecting into the second fluid pathway;
a third fluid pathway formed along the length of the second portion, the third fluid pathway comprising a third plurality of features projecting into the third fluid pathway; and
a fourth fluid pathway formed along the length of the second portion, the fourth fluid pathway comprising a fourth plurality of features projecting into the fourth fluid pathway; and
the first portion and the second portion configured to be independently fluidly coupled to the tissue interface.

2. The apparatus of claim 1, further comprising an interface coupled to the second end of the second portion and configured to fluidly couple the third fluid pathway and the fourth fluid pathway to a sensing pathway and the second fluid pathway to an instillation source.

3. The apparatus of claim 2, further comprising a connector configured to merge the first fluid pathway, the second fluid pathway, and the sensing pathway into a unibody object having a plurality of independent lumens.

4. The apparatus of claim 1, further comprising a connector configured to merge the first fluid pathway, the second fluid pathway, the third fluid pathway, and the fourth fluid pathway into a unibody object having a plurality of independent lumens.

5. The apparatus of claim 1, wherein the first portion and the second portion each comprise:
a first layer including a polymeric film having an outer surface, an inner surface, and the plurality of features extending from the inner surface;
a second layer including a polymeric film having an outer surface and an inner surface coupled to the first layer and covering the plurality of features to form a sealed space with the inner surface of the first layer and a plurality of flow channels within the sealed space, the second layer having an aperture disposed in the first end, open to the sealed space, and configured to fluidly couple the sealed space to the tissue interface; and
a port fluidly coupled to the second end.

6.-24. (canceled)

25. The apparatus of claim 1, wherein the first portion comprises:
a first layer having a first surface and a second surface;
a second layer having a first surface and a second surface;
an intermediate layer having a first surface and a second surface, a first plurality of features extending from the first surface and a second plurality of features extending from the second surface; and
the first surface of the first layer coupled to the first surface of the intermediate layer, and the first surface of the second layer coupled to the second surface of the intermediate layer.

26. The apparatus of claim 25, wherein the first plurality of features are offset from the second plurality of features.

27. The apparatus of claim 1, wherein the first fluid pathway, the second fluid pathway, the third fluid pathway, and the fourth fluid pathway comprise a manifolding layer disposed between two film layers.

28. (canceled)

29. The apparatus of claim 1, wherein the first end of the second portion fluidly couples the second fluid pathway, the third fluid pathway, and the fourth fluid pathway.

30. The apparatus of claim 1, wherein the first end of the second portion fluidly isolates the second fluid pathway, the third fluid pathway, and the fourth fluid pathway.

31. (canceled)

32. The apparatus of claim 1, wherein the first end of the second portion further comprises an aperture in fluid communication with the second fluid pathway, and a plurality of fenestrations in fluid communication with the third fluid pathway and the fourth fluid pathway, the aperture and the fenestrations covered by a removable cover layer.

33. An apparatus for managing fluid from a tissue site, the apparatus comprising:
a first bridge having:
a first layer including a polymeric film having an outer surface, an inner surface, and a first plurality of features extending from the inner surface;
a second layer including a polymeric film having an outer surface and an inner surface coupled to the first layer and covering the first plurality of features to form a first sealed space with the inner surface of the first layer and a first plurality of flow channels within the first sealed space, the second layer having an aperture configured to fluidly couple the first sealed space to the tissue site;
a first bridge conduit fluidly coupled to the first plurality of flow channels;
a second bridge having:
a third layer comprising a polymeric film and a second plurality of surface features extending from a surface of the third layer;
a fourth layer comprising a polymeric film, the fourth layer coupled to the third layer to cover the second plurality of surface features and to form a second sealed space between the third layer and the fourth layer; and
a first barrier and a second barrier coupled between the third layer and the fourth layer, wherein the first barrier and the second barrier define:
a second plurality of flow channels in the second sealed space between the first barrier and the second barrier;
a third plurality of flow channels in the second sealed space between the first barrier and a first seal formed between a first portion of the third layer and a first portion of the fourth layer; and
a fourth plurality of flow channels in the second sealed space between the second barrier and a second seal formed between a second portion of the third layer and a second portion of the fourth layer;
wherein the third plurality of flow channels and the fourth plurality of flow channels are outboard of the second plurality of flow channels;
a second bridge conduit fluidly coupled to the second plurality of flow channels; and
at least one sensing conduit fluidly coupled to the third plurality of flow channels and the fourth plurality of flow channels.

34. The apparatus of claim 33, further comprising a connector block configured to couple the first bridge conduit, the second bridge conduit and the at least one sensing conduit to each other.

35. The apparatus of claim 33, further comprising a slip ring configured to hold the first bridge conduit, the second bridge conduit, and the at least one sensing conduit to each other.

36. The apparatus of claim 33, wherein the at least one sensing conduit and the first bridge conduit are coupled to a multi-lumen conduit having a central lumen and a plurality of peripheral lumens, the peripheral lumens fluidly coupled to the at least one sensing conduit and the central lumen fluidly coupled to the first bridge conduit.

37. The apparatus of claim 36, wherein the multi-lumen conduit comprises a groove and the second bridge conduit comprises a tongue, the tongue configured to mate with the groove.

38.-40. (canceled)

41. A method of manufacturing an apparatus for managing fluid from a tissue site, the method comprising:
forming a first bridge comprising:
providing a first layer including a polymeric film having an outer surface, an inner surface, and a first plurality of features extending from the inner surface;
providing a second layer including a polymeric film having an outer surface and an inner surface;
coupling the inner surface of the second layer to the first layer and covering the first plurality surface of features to form a first sealed space with the inner surface of the first layer and a first plurality of flow channels within the first sealed space, the second layer having an aperture configured to fluidly couple the first sealed space to the tissue site;
fluidly coupling a first bridge conduit to the first plurality of flow channels;
forming a second bridge comprising:
providing a third layer comprising a polymeric film and a second plurality of features extending from a surface of the third layer;
providing a fourth layer comprising a polymeric film;
coupling the fourth layer to the third layer to cover the second plurality of surface features and to form a second sealed space between the third layer and the fourth layer; and
forming a first barrier and a second barrier between the third layer and the fourth layer, wherein the first barrier and the second barrier define:
a second plurality of flow channels in the second sealed space between the first barrier and the second barrier;
a third plurality of flow channels in the second sealed space between the first barrier and a first seal formed between a first portion of the third layer and a first portion of the fourth layer; and
a fourth plurality of flow channels in the second sealed space between the second barrier and a second seal formed between a second portion of the third layer and a second portion of the fourth layer;
wherein the third plurality of flow channels and the fourth plurality of flow channels are outboard of the second plurality of flow channels;
fluidly coupling a second bridge conduit to the second plurality of flow channels; and
fluidly coupling at least one sensing conduit to the third plurality of flow channels and the fourth plurality of flow channels.

42. The method of claim 41, wherein the method further comprises coupling the first bridge conduit, the second bridge conduit and the at least one sensing conduit to each other with a connector block.

43. The method of claim 41, wherein the method further comprises providing a slip ring configured to hold the first bridge conduit, the second bridge conduit, and the at least one sensing conduit to each other.

44. (canceled)

45. (canceled)

46. The method of claim 41, wherein the method further comprises forming a plurality of tabs along a length of the first bridge; and releasably coupling the second bridge to the tabs.

47. (canceled)
